(19) United States
(12) Patent Application Publication
(10) Pub. No.: US 2014/0363456 A1
(43) Pub. Date: Dec. 11, 2014

(54) TUMOR-SPECIFIC GM-CSF CYTOKINE RESPONSE AS PREDICTOR OF CANCER VACCINE EFFECTIVENESS

(71) Applicants: BIOVEST INTERNATIONAL, INC., TAMPA, FL (US); THE UNITED STATES OF AMERICA AS REPRESENTED BY THE SECRETARY, DEPARTMENT OF HEALTH AND HUMAN SERVICE, BETHESDA, MD (US)

(72) Inventors: Wyndham H. Wilson, Washington, DC (US); Carlos Santos, Minneapolis, MN (US); Larry W. Kwak, Bellaire, TX (US); Sattva S. Neelapu, Pearland, TX (US)

(21) Appl. No.: 14/362,093

(22) PCT Filed: Dec. 7, 2012
(86) PCT No.: PCT/US2012/068574

§ 371 Date: May 30, 2014

Related U.S. Application Data
(60) Provisional application No. 61/569,131, filed on Dec. 9, 2011.

(51) Int. Cl. A61K 39/00; G01N 33/68

(52) U.S. Cl. CPC A61K 39/0011; G01N 33/6863; G01N 2333/535
USPC 424/184.1; 435/7.1; 435/7.92

(57) ABSTRACT

The present invention relates to methods of treating cancer with a cancer vaccine using granulocyte-macrophage colony-stimulating factor (GM-CSF) as a biomarker; methods of prognosticating an outcome of cancer treatment with a cancer vaccine using GM-CSF as a biomarker; methods for qualifying subjects for cancer vaccination using GM-CSF as a biomarker; methods for comparing the efficacy of two or more cancer vaccine treatments based on GM-CSF response; and kits for the effective treatment of cancer.

### CROSS-REFERENCE TO RELATED APPLICATION

The present application claims the benefit of U.S. Provisional Application Ser. No. 61/569,131, filed Dec. 9, 2011, which is hereby incorporated by reference herein in its entirety, including any figures, tables, or drawings.

### BACKGROUND OF THE INVENTION

Surgery, chemotherapy and radiation therapy are the mainstay of cancer treatment and management. Surgery and radiation therapy are typically used to achieve results locally, whereas chemotherapy exerts a more systemic effect. However, usually, remaining cancer cells are able to divide, thereby leading to a relapse of the cancer. Accordingly, despite the use of combination chemotherapy to treat various types of cancers, a significant number of cancers remain incurable.

Immunotherapeutic strategies have recently emerged to become the latest addition to the toolbox of cancer treatments. The central premise underlying immunotherapy for cancer is the presence of antigens that are selectively or abundantly expressed or mutated in cancer cells. Tumor-specific immunotherapies can be classified into passive immunotherapy with antibodies targeted directly to tumor cells or active immunotherapy via vaccination with tumor cells, tumor cell lysates, peptides, carbohydrates, genetic constructs encoding proteins, or anti-idiotypic antibodies that mimic tumor-associated antigens (TAA).

Although some cancer patients can be effectively treated using current methods, there remains a need for biological markers (biomarkers) that would improve prognosis and treatment of cancer patients. The term biomarker refers to any biological indicator that reflects disease status but may not necessarily be involved in the disease process itself. In some patients, the responses to cancer immunotherapy are durable, providing dramatically extended survival. Research efforts are being made to identify and validate biomarkers that can help identify subsets of cancer patients that will benefit most from these novel immunotherapies. In addition to the advantage of such predictive biomarkers, immune biomarkers are playing an important role in the development, clinical evaluation and monitoring of cancer immunotherapies. Biomarkers can aid patient selection by allowing stratification and risk assessment and can potentially predict treatment responses based on the presence or extent of surrogate markers. Clinical endpoints such as overall survival (OS) and time to disease progression (TTP) can require very long and expensive clinical trial protocols. Accurate biomarkers could reduce the time needed to assess if a drug has significant clinical activity in Phase II trials before progressing to larger and more costly Phase III trials. In addition to disease-specific biomarkers, there are immune response biomarkers that may be of use in identifying patients who are developing an effective clinical response at an early stage. It would be advantageous to select these patients as early as possible, and to provide alternative treatment modalities to non-responding patients (Whelan M. et al., “Biomarkers For Development of Cancer Vaccines,” Personalized Medicine, 2006, 3(1):79-88).

### BRIEF SUMMARY OF THE INVENTION

Following the introduction and increased use of monoclonal antibodies (mAb) (e.g., rituximab) for the treatment of B-cell malignancies, the NCI conducted a Phase 2 trial in MCL patients who received an immunochemotherapy induction regimen (etoposide, vincristine, doxorubicin, cyclophosphamide, prednisone, rituximab: EPOCH-R) prior to vaccine administration to investigate whether specific immune responses are mounted in the context of almost complete B-cell depletion. The Phase 2 trial titled “Pilot Study of Idiotype Vaccine and EPOCH-R Chemotherapy in Untreated Mantle Cell Lyphoma (NCI1033, NCT00020215)” investigated the BiovaxID patient-specific anti-idiotype vaccine in MCL patients. These results were published by Neelapu, S. S., Kwak, L. W., et al. (September 2005) “Vaccine-induced tumor-specific immunity despite severe B-cell depletion in mantle cell lymphoma”, Nat Med 11.9, pp. 986-91.

This study, together with a recently-reported long-term follow-up, demonstrates the activity of Id vaccination following rituximab-containing combination chemotherapy. Importantly, as of 2011, with 122 months of median potential follow-up (range 111-132 months), the median PFS is 24 months and OS is 104 months. This overall survival exceeds the reported OS for low-risk MIPI patients diagnosed with MCL historically and substantially exceeds that reported historically for “high-risk” or “intermediate-risk” MIPI. In the present study, MIPI was associated with OS (p=0.01); median OS: low (not reached), interim (84 months) and high (44 months).

There was a significant association between antitumor GM-CSF production and both OS and time-to-next-treatment correlating with vaccine-specific GM-CSF response. The median OS at the median GM-CSF normalized value (<4.3 years versus >4.3) was 79 mos vs not yet reached, respectively (p=0.015 (unadjusted) and p=0.045 (bonferroni adjusted)). MIPI and GM-CSF were jointly assessed in a Cox model and showed a trend toward improved OS for higher GM-CSF (p=0.10) after adjusting for MIPI (p=0.20). There was no association between OS and KLH humoral response or KLH-specific CD4+ T cells in this study after long-term follow-up. These findings suggest that immune competence as a whole did not correlate with OS. Moreover, no association was found between OS and Id-specific humoral response, IFNγ ELISPOT, antitumor TNFα, or IFNγ cytokine responses.

The invention, in its various aspects, pertains to the use of tumor-specific (anti-tumor) granulocyte-macrophage colony-stimulating factor (GM-CSF) cytokine response as a biomarker to predict the effectiveness of cancer vaccines. One aspect of the invention concerns a method of prognosticating an outcome of cancer treatment with a cancer vaccine in a subject, comprising comparing the level of tumor-specific GM-CSF in a sample obtained from the subject with a reference level of GM-CSF, wherein the level of tumor-specific GM-CSF in the sample compared to the reference level of GM-CSF is prognostic for an outcome of treatment with the cancer vaccine. The clinical outcome of treatment may be, for example, overall survival (OS), time-to-next-treatment (TNTT), time from first progression to next treatment, or a combination of two or more of the foregoing. The ability to predict clinical outcome facilitates effective treatment of cancer. Thus, if it is determined that the currently administered regimen is inadequate, it can be modified (e.g., by frequency of administration and/or dosage) or a different treatment may be selected for the subject. Likewise, if a positive clinical outcome is predicted, the treatment regimen may be maintained. Other prognostic markers, in conjunction with tumor-specific GM-CSF, may be utilized.

Another aspect of the invention concerns a method for treating cancer in a subject with a cancer vaccine, the method comprising: assessing the level of tumor-specific GM-CSF in a sample obtained from a subject that has been administered a cancer vaccine for treatment of a cancer; and determining whether the level of tumor-specific GM-CSF has diminished in the subject. In some embodiments, the method further comprises administering at least one booster dose of cancer vaccine to the subject if the level of tumor-specific GM-CSF is determined to have diminished. In some embodiments, the assessing and determining are carried out multiple times over time, and the one or more booster doses of the cancer vaccine are administered to the subject as needed. In some embodiments, the sample is obtained from the subject immediately after administration of the cancer vaccine, and, optionally, within 3 days after the first administration of the cancer vaccine. The determining step may comprise determining whether the level of tumor-specific GM-CSF has diminished to an extent that is inconsistent with a positive clinical outcome (e.g., increase in overall survival). In some embodiments, the positive clinical outcome is selected from alleviation of one or more symptoms of the cancer, diminishment of extent of disease, stabilized (i.e., not worsening) state of disease, delay or slowing of disease progression, amelioration or palliation of the disease state, remission (whether partial or total), whether detectable or undetectable, tumor regression, inhibition of tumor growth, inhibition of tumor metastasis, reduction in cancer cell number, inhibition of cancer cell infiltration into peripheral organs, improved time to disease progression (TTP), improved response rate (RR), prolonged overall survival (OS), prolonged time-to-next-treatment (TNTT), or prolonged time from first progression to next treatment, or a combination of two or more of the foregoing.

Another aspect of the invention concerns a kit for the effective treatment of cancer, comprising: one or more doses of a cancer vaccine; and one or more reagents for the detection of tumor-specific GM-CSF. In some embodiments, the one or more reagents comprise an antibody directed against GM-CSF or an oligonucleotide probe targeting GM-CSF DNA or mRNA.

Kits of the invention may comprise packaging and containers or receptacles for containing each component of the kit. The kits can also contain a solid support such as microtiter multi-well plates, standards, assay diluent, wash buffer, adhesive plate covers, and/or instructions for carrying out a method of the invention using the kit. If a biological sample is to be obtained (such as for an assay for a tumor-specific GM-CSF response), the kit can include means for obtaining a biological sample (such as a needle for venipuncture) and/or one or more protease inhibitors (e.g., a protease inhibitor cocktail) to be applied to the biological sample to be assayed (such as blood).

Another aspect of the invention concerns a method for qualifying subjects for cancer vaccination, the method comprising assessing the level of GM-CSF in a sample obtained from one or more subjects, wherein cancer vaccination is authorized if the level of GM-CSF in the sample is consistent with effective treatment, and wherein cancer vaccination is not authorized if the level of GM-CSF in the sample is not consistent with effective treatment. In some embodiments, the subject has cancer and the subject is under consideration for administration of a cancer vaccine, wherein administration of the cancer vaccine is authorized if the level of GM-CSF in the sample is consistent with effective treatment for the cancer, and wherein administration of the cancer vaccine is not authorized if the level of GM-CSF in the sample is not consistent with effective treatment for the cancer.

Another aspect of the invention concerns a method for treating cancer in a subject by inducing a GM-CSF response against a tumor-specific antigen (TSA) or tumor-associated antigen (TAA) in the subject, the method comprising administering an effective amount of the TSA or TAA to the subject to induce the GM-CSF response. In some embodiments, the treatment method further comprises assessing the GM-CSF response against the TSA or TAA in the subject one or more times before, during, and/or after administering the TSA or TAA to the subject.

Another aspect of the invention concerns a method for comparing the efficacy of two or more cancer vaccine treatments. The anti-tumor GM-CSF response may be used as a surrogate to compare the efficacy of different cancer vaccine treatments that differ from each other with respect to composition, with respect to treatment regimen, or both. For example, depending upon the particular comparison, the compositions of the vaccines may differ with respect to one or more of the following parameters: a) tumor antigens; b) formulation (e.g., differences in diluent); c) carrier protein/vehicle (e.g., KLH versus liposomes); or d) adjuvants (e.g., IL-2, TLR ligands, GM-CSF). The anti-tumor GM-CSF response may allow rapid comparison of different vaccine formulations to select the best candidate for further clinical development. The same strategy may also be used for comparing the efficacy of cancer vaccine regimens. For example, the efficacy of different doses and/or frequency of cancer vaccines may be compared.

In the methods and kits of the invention, the sample may be any biological sample (fluid, tissue, etc.) in which GM-CSF may be found and the level determined. Preferably, the sample comprises or consists of helper T-cells or supernatant from a culture of the helper T-cells.

In the methods and kits of the invention, a reference level of tumor-specific GM-CSF may be used as a comparison to the level of tumor-specific GM-CSF in a sample. The reference level of GM-CSF may represent a statistical mean of a population of subjects with or without cancer. The reference level of GM-CSF may serve as a control. The reference level of GM-CSF may be a level of GM-CSF in a sample previously obtained from the same subject before treatment with the cancer vaccine, during treatment with the cancer vaccine, or after treatment with the cancer vaccine. The reference level of GM-CSF may be a range, threshold, cutoff, or other value, or a symbol representative thereof.

In some embodiments, the subject has been previously treated with the cancer vaccine before the sample is obtained from the subject. In some embodiments, the subject has been previously treated with the cancer vaccine, and the method further comprises administering a different treatment for the cancer if the prognosticated outcome of treatment with the cancer vaccine is not desirable.

In the methods of the invention, the level of tumor-specific GM-CSF in a subject (also referred to herein as the tumor-specific GM-CSF response) may be monitored over time. In some embodiments, a sample is obtained from the subject immediately before and/or immediately after the first administration of the cancer vaccine (e.g., within day 0 to day+5), to establish a steady state tumor-specific GM-CSF level, and again within 30 days, 60 day, 90 day, or 180 days after the first administration of the cancer vaccine, and assessed for GM-CSF. In some embodiments, the subject has not been treated with the cancer vaccine before the sample is obtained from the subject for assessment. For example, if an induction regimen (e.g., immunochemotherapy) is first used to place the subject in complete remission, a sample can be obtained after induction and again immediately before and/or immediately after administration of the cancer vaccine, and assessed for tumor-specific GM-CSF. In some embodiments, a sample is obtained from the subject and assessed for tumor-specific GM-CSF level after every vaccination. Changes in the tumor-specific GM-CSF level pre-vaccination (or immediately after vaccination) and post-vaccination (e.g., at 30 days, 60 days, 90 days, 180 days, and so forth) can be monitored. If the level of tumor-specific GM-CSF falls below, or does not reach a level (threshold or range) that is consistent with a positive clinical outcome, the frequency and/or dose of vaccination can be increased and the level of tumor-specific GM-CSF monitored further until the level of tumor-specific GM-CSF reaches or exceeds a level (threshold or range) that is consistent with a positive clinical outcome.

In some embodiments, the methods further comprises the step of determining the level of tumor-specific GM-CSF in the sample prior to comparing the level of tumor-specific GM-CSF in the sample to the reference level of tumor-specific GM-CSF. The level of tumor-specific GM-CSF may be determined at the protein level using an immunoassay. In some embodiments, the level of tumor-specific GM-CSF is determined using a competitive or immunometric assay, such as a radioimmunoassay (RIA), immunoradiometric assay (IRMA), enzyme-linked immunosorbent assay (ELISA), or enzyme-linked immunosorbent spot (ELISPOT) assay.

The level of tumor-specific GM-CSF is determined by determining the level of tumor-specific GM-CSF mRNA or the level of tumor-specific GM-CSF protein. The level of tumor-specific GM-CSF may be determined, for example, by using surface plasmon resonance, fluorescence resonance energy transfer, bioluminescence resonance energy transfer, fluorescence quenching fluorescence, fluorescence polarization, mass spectrometry (MS), high-performance liquid chromatography (HPLC), high-performance liquid chromatography/mass spectrometry (HPLC/MS), high-performance liquid chromatography/mass spectrometry/mass spectrometry (HPLC/MS/MS), capillary electrophoresis, rod-gel electrophoresis, or slab-gel electrophoresis.

The methods of the invention may further include the step of obtaining the sample from the subject prior to comparing the level of tumor-specific GM-CSF in the sample to the reference GM-CSF level. Obtaining the sample from the subject and comparing GM-CSF levels can be carried out by the same party or different parties.

In the methods and kits of the invention, potentially any cancer may be treated depending upon the type of cancer vaccine utilized. In some embodiments, the cancer is a B-cell malignancy. In some embodiments, the B-cell malignancy is selected from the group consisting of non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, Burkitt lymphoma/leukemia. In some embodiments, the B-cell malignancy is a mature B-cell lymphoma. In some embodiments, the B-cell malignancy is a mature B-cell lymphoma selected from the group consisting of B-cell chronic lymphocytic leukemia/small lymphocytic lymphoma, B-cell prolymphocytic leukemia, lymphoplasmacytic lymphoma, splenic marginal zone B-cell lymphoma (½ villous lymphocytes), hairy cell leukemia, plasma cell myeloma/plasmacytoma, extranodal marginal zone B-cell lymphoma of MALT type, nodal marginal zone B-cell lymphoma (½ monocytoid B cells), follicular lymphoma, mantle-cell lymphoma, diffuse large B-cell lymphoma, mediastinal large B-cell lymphoma, primary effusion lymphoma, Burkitt lymphoma/Burkitt cell leukemia. In some embodiments, the B-cell malignancy is mantle cell lymphoma or follicular lymphoma. In some embodiments, the cancer is a B-cell malignany, such as mantle cell lymphoma, follicular lymphoma, or another B-cell malignancy, and the cancer vaccine is an autologous idiotype vaccine (e.g., a hybridoma-derived autologous idiotype vaccine).

In the methods of the invention, various cancer vaccines may potentially be utilized to treat the cancer. In some embodiments, the cancer vaccine is an autologous idiotype vaccine, such as a hybridoma-derived idiotype vaccine or a recombinant idiotype vaccine. In some embodiments, the cancer vaccine is selected from among a peptide vaccine, plasmid DNA vaccine, recombinant viral vector, recombinant bacteria, dendritic cell vaccine, tumor cell vaccine, heat-shock protein, or exosome-based vaccine.

In the methods of the invention, the cancer treatment may further comprise an additional (different) cancer treatment. For example, the additional cancer treatment may include chemotherapy, radiation, immunotherapy, or a combination of two or more of the foregoing.

In some embodiments of the methods of the invention, the subject has previously undergone a different therapy for treatment of the cancer. In some embodiments, the different therapy comprises chemotherapy and/or immunotherapy. In some embodiments, the different therapy comprises administration of a monoclonal antibody (e.g., anti-CD20 antibody, such as rituximab). In some embodiments, the different therapy comprises a radioimmunotherapy. In some embodiments, the different therapy comprises a regimen of PACE (prednisone, doxorubicin, cyclophosphamide, and etoposide), CHOP (cyclophosphamide, doxorubicin, vincristine, and prednisone), CHOP-R (cyclophosphamide, doxorubicin, vincristine, prednisone, rituximab), B-R (bendamustine and rituximab), CVP (cyclophosphamide, vincristine, and prednisone), CVP-R (cyclophosphamide, vincristine, prednisone, and rituximab), F-R (fluradarabine and rituximab), FND-R (fludarabine, mitoxantrone, dexamethasone, and rituximab), FCM (fludarabine, cyclophosphamide, and mitoxantrone), FCM-R (fludarabine, cyclophosphamide, mitoxantrone, and rituximab), radioimmunotherapy, single agent rituximab, single agent alkylator, lenalidomide, involved field radiation therapy, or stem cell transplant.

In some embodiments, the subject has undergone an immunochemotherapy induction regimen for treatment of the cancer, such as a regimen comprising administration of a monoclonal antibody directed against CD20 antigen, and chemotherapy. In some embodiments, the monoclonal antibody is rituximab. In some embodiments, the immunochemotherapy induction regimen is a rituximab-containing combination chemotherapy comprises or consists of etoposide, vincristine, doxorubicin, cyclophosphamide, prednisone, and ritruximab (EPOCH-R).

In some embodiments, the subject is in complete remission at the time the cancer vaccine is administered to the subject. In some embodiments, the subject is not in complete remission at the time the cancer vaccine is administered to the subject.

In the methods of the invention, the subject may be human or a non-human animal, such as an animal model.

### BRIEF DESCRIPTION OF THE FIGURES

FIG. 1 shows overall survival (all patients enrolled) for the MCL phase 2 Id vaccine study.

FIG. 2 shows overall survival by MIPI score. With 122 months median potential follow-up (range: 111-132), the median PFS is 24 months and OS is 104 months. MIPI was associated with OS (p=0.015); median OS: low (not reached), intermediary (84 months) and high (44 months).

FIG. 3 shows overall survival (Id-specific humoral response). Overall Survival (Tumor-specific anti-Id antibody < and > than median):

There was no association between OS and KLH humoral response or KLH-specific CD4+ T cells, or between OS and Id-specific humoral response, IFNγ ELISPOT, antitumor TNFα, or IFNγ cytokine responses. These results suggest that OS and TTNT benefit do not depend on Id-specific humoral response.

FIGS. 4A-4D show time-to-next treatment and overall survival. FIG. 4A: Time-to-Next Treatment (Tumor-specific anti-Id antibody < and > than median). FIG. 4B: Time-To-Next Treatment (Non-Specific anti-KLH antibody < and > than median). FIG. 4C: Overall Survival (Tumor-specific anti-Id antibody < and > than median). FIG. 4D: Overall Survival (Non-specific anti-KLH antibody < and > than median) in MCL Phase 2 Clinical Trial. There was no association between OS and KLH humoral response or KLH-specific CD4+ T cells, or between OS and Id-specific humoral response, IFNγ ELISPOT, antitumor TNFα, or IFNγ cytokine responses. These results suggest that OS and TTNT benefit do not depend on Id-specific humoral response nor does the benefit depend on non-specific, but robust immune responses. Only Id-specific GM-CSF cytokine response correlated with both OS and TTNT.

FIG. 5 shows time-to-next treatment (tumor-specific GM-CSF response < and > than median).

FIG. 6 shows overall survival (tumor-specific GM-CSF < and > than median).

FIG. 7 shows associations between overall survival (OS) and MIPI scores.

FIG. 8 shows significant association between antitumor GM-CSF production and overall survival (OS).

### DETAILED DESCRIPTION OF THE INVENTION

It was hypothesized that immunotherapy with autologous tumor-derived idiotype (Id)-vaccine may improve the outcome of mantle cell lymphoma (MCL). Some murine lymphoma models have shown that Id-vaccine can induce an anti-tumor humoral response but others indicate that eradication of tumor requires a CD4+ and/or a CD8+ T-cell response. Antitumor T-cells may produce one or many cytokines. The Thl/Tcl cytokines (IFNγ, IL-2, TNFα, GM-CSF) are commonly believed to mediate antitumor effects. However, a recent paper (Codarri et al. Nat Immunol, 2011) proposes that production of GM-CSF by helper T-cells relies on activation of RORγt and that GM-CSF secretion is required for induction of autoimmune inflammation irrespective of helper T-cell polarization. The results of Id-vaccine following DA-EPOCH-R in 26 untreated MCL patients were reported (Neelapu et al. Nat Med, 2005) and found no association between PFS (19%) or OS (89%) and immune responses at the median of 46 months potential follow-up. We now present an 11-year follow-up and association between OS and antitumor immune responses.

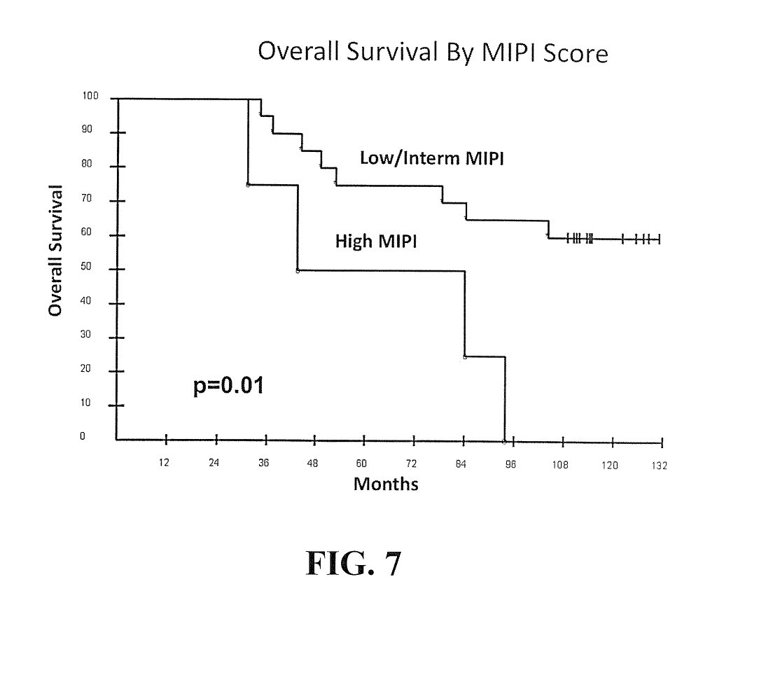

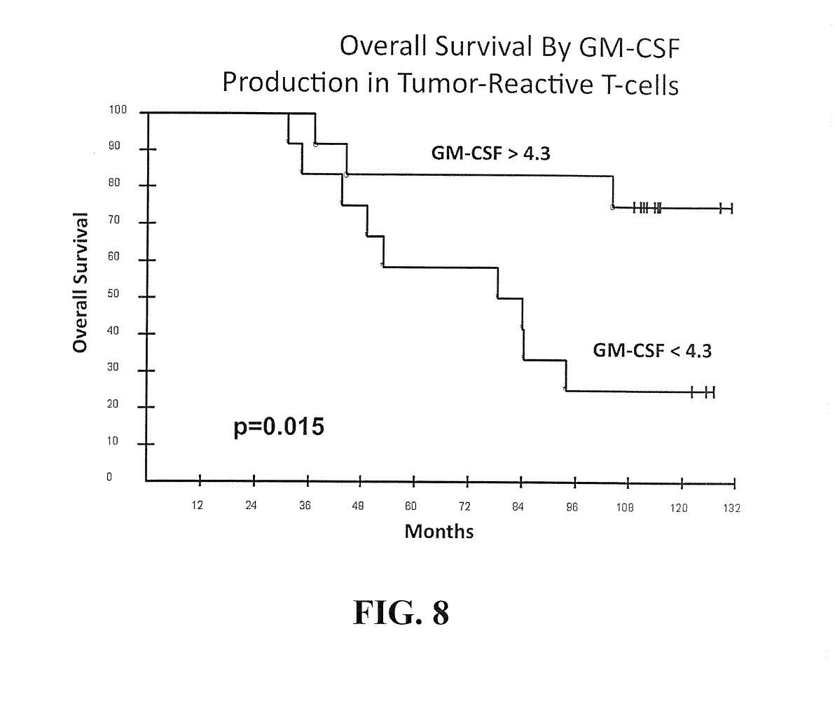

DA-EPOCH-R was administered q3 weeks×6, followed by 5 cycles of Id-vaccine beginning at least 12 weeks later to untreated MCL patients. Id protein was produced using hybridoma technology, conjugated to keyhole limpet hemocyanin (KLH), and administered together with GM-CSF×5 over 6 months. Pre- and post-vaccine samples were tested in parallel to assess humoral and cellular immune responses. Anti-Id and anti-KLH antibody responses were determined by ELISA. KLH-specific cellular responses were determined by intracellular cytokine assay and cellular responses against autologous tumor cells were determined by cytokine induction and IFNγ ELISPOT assays. For cytokine induction assay, PBMCs were cultured with and without autologous tumor cells. After 6 days TNFα, IFNγ and GM-CSF were assessed in culture supernatants by ELISA. Normalized post-vaccine responses were calculated for each patient. Results: Characteristics of all 26 patients: median age 57 (r 22-73), PS 1 (0-2), male sex 73%, blastoid variant 15%, and MIPI (low-65%; intermediate-16%; high-19%). Responses to DA-EPOCH-R: CR-92%, PR-8%. Immune analyses were performed in 24 patients; vaccine could not be made in one patient and one patient progressed and did not have immune analyses. The associations between OS and MIPI scores and normalized immune responses (KLH and anti-Id antibody responses, frequency of KLH-specific CD4+ T-cell responses in PBMC (intracellular IL-2 and TNFα), antitumor cytokine responses and IFNγ ELISPOT) were determined. With 122 mos median potential follow-up (r 111-132), the median PFS is 24 mos and OS is 104 mos. MIPI was significantly associated with OS (FIG. 7; p=0.01); median OS: low (not reached), intermediate (84 mos) and high (44 mos). There was no association between OS and KLH humoral response or KLH-specific CD4+ T cells. There was also no association between OS and Id-specific humoral response, IFNγ ELISPOT, or antitumor TNFα, or IFNγ cytokine responses. However, there was a significant association between antitumor GM-CSF production and OS (FIG. 8). The median OS at the median GM-CSF normalized value (<4.3 versus >4.3) was 79 mos versus not reached, respectively (p=0.015 (unadjusted) and p=0.045 (bonferroni adjusted)). MCL international prognostic index (MIPI) and GM-CSF were jointly assessed in a Cox model and showed a trend toward improved OS for higher GM-CSF (p=0.10) after adjusting for MIPI (p=0.20). Conclusions: With 10-year median potential follow-up, GM-CSF cytokine response mediated by antitumor T-cells was significantly associated with OS. Recent studies support the hypothesis that antitumor T-cells that produce significant amounts of GM-CSF are uniquely polarized and that non-GM-CSF producing T-cells do not induce antitumor effects even if they produce TNFα or IFNγ. This may explain why we did not observe an association between OS and TNFα or IFNγ cytokine responses or an anti-Id-antibody response. These results provide the first evidence that Id-vaccines may improve the survival of MCL following induction with immuno-chemotherapy.

One aspect of the invention concerns a method of prognosticating an outcome of cancer treatment with a cancer vaccine in a subject, comprising comparing the level of tumor-specific GM-CSF in a sample obtained from the subject with a reference level of GM-CSF, wherein the level of tumor-specific GM-CSF in the sample compared to the reference level of GM-CSF is prognostic for an outcome of treatment with the cancer vaccine. The clinical outcome of treatment may be, for example, overall survival (OS), time-to-next-treatment (TNTT), time from first progression to next treatment, or a combination of two or more of the foregoing. The ability to predict clinical outcome facilitates effective treatment of cancer. Thus, if it is determined that the currently administered regimen is inadequate, it can be modified (e.g., by frequency of administration and/or dosage) or a different treatment may be selected for the subject. Likewise, if a positive clinical outcome is predicted, the treatment regimen may be maintained. Other prognostic markers, in conjunction with tumor-specific GM-CSF, may be utilized.

Another aspect of the invention concerns a method for treating cancer in a subject with a cancer vaccine, the method comprising: assessing the level of tumor-specific GM-CSF in a sample obtained from a subject that has been administered a cancer vaccine for treatment of a cancer; and determining whether the level of tumor-specific GM-CSF has diminished in the subject. In some embodiments, the method further comprises administering at least one booster dose of cancer vaccine to the subject if the level of tumor-specific GM-CSF is determined to have diminished. In some embodiments, the assessing and determining are carried out multiple times over time, and the one or more booster doses of the cancer vaccine are administered to the subject as needed. In some embodiments, the sample is obtained from the subject immediately after administration of the cancer vaccine, and, optionally, within 3 days after the first administration of the cancer vaccine. The determining step may comprise determining whether the level of tumor-specific GM-CSF has diminished to an extent that is inconsistent with a positive clinical outcome (e.g., increase in overall survival). In some embodiments, the positive clinical outcome is selected from alleviation of one or more symptoms of the cancer, diminishment of extent of disease, stabilized (i.e., not worsening) state of disease, delay or slowing of disease progression, amelioration or palliation of the disease state, remission (whether partial or total), whether detectable or undetectable, tumor regression, inhibition of tumor growth, inhibition of tumor metastasis, reduction in cancer cell number, inhibition of cancer cell infiltration into peripheral organs, improved time to disease progression (TTP), improved response rate (RR), prolonged overall survival (OS), prolonged time-to-next-treatment (TNTT), or prolonged time from first progression to next treatment, or a combination of two or more of the foregoing.

Another aspect of the invention concerns a kit for the effective treatment of cancer, comprising: one or more doses of a cancer vaccine; and one or more reagents for the detection of tumor-specific GM-CSF. In some embodiments, the one or more reagents comprise an antibody directed against GM-CSF or an oligonucleotide probe targeting GM-CSF DNA or mRNA.

Kits of the invention may comprise packaging and containers or receptacles for containing each component of the kit. The kits can also contain a solid support such as microtiter multi-well plates, standards, assay diluent, wash buffer, adhesive plate covers, and/or instructions for carrying out a method of the invention using the kit. If a biological sample is to be obtained (such as for an assay for a tumor-specific GM-CSF response), the kit can include means for obtaining a biological sample (such as a needle for venipuncture) and/or one or more protease inhibitors (e.g., a protease inhibitor cocktail) to be applied to the biological sample to be assayed (such as blood).

Another aspect of the invention concerns a method for qualifying subjects for cancer vaccination, the method comprising assessing the level of GM-CSF in a sample obtained from one or more subjects, wherein cancer vaccination is authorized if the level of GM-CSF in the sample is consistent with effective treatment, and wherein cancer vaccination is not authorized if the level of GM-CSF in the sample is not consistent with effective treatment. In some embodiments, the subject has cancer and the subject is under consideration for administration of a cancer vaccine, wherein administration of the cancer vaccine is authorized if the level of GM-CSF in the sample is consistent with effective treatment for the cancer, and wherein administration of the cancer vaccine is not authorized if the level of GM-CSF in the sample is not consistent with effective treatment for the cancer.

Another aspect of the invention concerns a method for treating cancer in a subject by inducing a GM-CSF response against a tumor-specific antigen (TSA) or tumor-associated antigen (TAA) in the subject, the method comprising administering an effective amount of the TSA or TAA to the subject to induce the GM-CSF response. In some embodiments, the treatment method further comprises assessing the GM-CSF response against the TSA or TAA in the subject one or more times before, during, and/or after administering the TSA or TAA to the subject.

Another aspect of the invention concerns a method for comparing the efficacy of two or more cancer vaccine treatments. The anti-tumor GM-CSF response may be used as a surrogate to compare the efficacy of different cancer vaccine treatments that differ from each other with respect to composition, with respect to treatment regimen, or both. For example, depending upon the particular comparison, the compositions of the vaccines may differ with respect to one or more of the following parameters: a) tumor antigens; b) formulation (e.g., differences in diluent); c) carrier protein/vehicle (e.g., KLH versus liposomes); or d) adjuvants (e.g., IL-2, TLR ligands, GM-CSF). The anti-tumor GM-CSF response may allow rapid comparison of different vaccine formulations to select the best candidate for further clinical development. The same strategy may also be used for comparing the efficacy of cancer vaccine regimens. For example, the efficacy of different doses and/or frequency of cancer vaccines may be compared. Accordingly, an aspect of the invention includes a method for comparing the efficacy of two or more cancer vaccine treatments, comprising comparing the level of tumor-specific (anti-tumor) granulocyte macrophage colony-stimulating factor (GM-CSF) response from a first subject that has received a first cancer vaccine treatment to the level of GM-CSF response from a second subject that has received a second cancer vaccine treatment, wherein the first vaccine cancer treatment and the second cancer vaccine treatment differ in cancer vaccine composition and/or in cancer vaccine administration. The levels of GM-CSF response under comparison may be corrected or normalized by first comparing the GM-CSF response to a reference level or control. In some embodiments, the first subject and the second subject are the same subject. In other embodiments, the first subject and the second subject are different subjects (preferably, different subjects of the same species). In some embodiments, the first cancer vaccine and the second cancer vaccine differ from each other with respect to at least tumor antigen, formulation, carrier molecule/vehicle, or adjuvant. In some embodiments, the first cancer vaccine and the second cancer vaccine differ from each other in dose and/or frequency. In some embodiments, the level of tumor-specific GM-CSF response is obtained from a sample obtained from the first subject and the level of tumor-specific GM-CSF response is obtained from a sample obtained from the second subject.

In the methods and kits of the invention, the sample may be any biological sample (fluid, tissue, etc.) in which GM-CSF may be found and the level determined. Preferably, the sample comprises or consists of helper T-cells or supernatant from a culture of the helper T-cells.

In the methods and kits of the invention, a reference level of tumor-specific GM-CSF may be used as a comparison to the level of tumor-specific GM-CSF in a sample. The reference level of GM-CSF may represent a statistical mean of a population of subjects with or without cancer. The reference level of GM-CSF may serve as a control. The reference level of GM-CSF may be a level of GM-CSF in a sample previously obtained from the same subject before treatment with the cancer vaccine, during treatment with the cancer vaccine, or after treatment with the cancer vaccine. The reference level of GM-CSF may be a range, threshold, cutoff, or other value, or a symbol representative thereof.

In some embodiments, the subject has been previously treated with the cancer vaccine before the sample is obtained from the subject. In some embodiments, the subject has been previously treated with the cancer vaccine, and the method further comprises administering a different treatment for the cancer if the prognosticated outcome of treatment with the cancer vaccine is not desirable.

In the methods of the invention, the level of tumor-specific GM-CSF in a subject (also referred to herein as the tumor-specific GM-CSF response) may be monitored over time. In some embodiments, a sample is obtained from the subject immediately before and/or immediately after the first administration of the cancer vaccine (e.g., within day 0 to day+5), to establish a steady state tumor-specific GM-CSF level, and again within 30 days, 60 day, 90 day, or 180 days after the first administration of the cancer vaccine, and assessed for GM-CSF. In some embodiments, the subject has not been treated with the cancer vaccine before the sample is obtained from the subject for assessment. For example, if an induction regimen (e.g., immunochemotherapy) is first used to place the subject in complete remission, a sample can be obtained after induction and again immediately before and/or immediately after administration of the cancer vaccine, and assessed for tumor-specific GM-CSF. In some embodiments, a sample is obtained from the subject and assessed for tumor-specific GM-CSF level after every vaccination. Changes in the tumor-specific GM-CSF level pre-vaccination (or immediately after vaccination) and post-vaccination (e.g., at 30 days, 60 days, 90 days, 180 days, and so forth) can be monitored. If the level of tumor-specific GM-CSF falls below, or does not reach a level (threshold or range) that is consistent with a positive clinical outcome, the frequency and/or dose of vaccination can be increased and the level of tumor-specific GM-CSF monitored further until the level of tumor-specific GM-CSF reaches or exceeds a level (threshold or range) that is consistent with a positive clinical outcome.

In some embodiments, the methods further comprises the step of determining the level of tumor-specific GM-CSF in the sample prior to comparing the level of tumor-specific GM-CSF in the sample to the reference level of tumor-specific GM-CSF. The level of tumor-specific GM-CSF may be determined at the protein level using an immunoassay. In some embodiments, the level of tumor-specific GM-CSF is determined using a competitive or immunometric assay, such as a radioimmunoassay (RIA), immunoradiometric assay (IRMA), enzyme-linked immunosorbent assay (ELISA), or enzyme-linked immunosorbent spot (ELISPOT) assay.

The level of tumor-specific GM-CSF is determined by determining the level of tumor-specific GM-CSF mRNA or the level of tumor-specific GM-CSF protein. The level of tumor-specific GM-CSF may be determined, for example, by using surface plasmon resonance, fluorescence resonance energy transfer, bioluminescence resonance enemy transfer, fluorescence quenching fluorescence, fluorescence polarization, mass spectrometry (MS), high-performance liquid chromatography (HPLC), high-performance liquid chromatography/mass spectrometry (HPLC/MS), high-performance liquid chromatography/mass spectrometry/mass spectrometry (HPLC/MS/MS), capillary electrophoresis, rod-gel electrophoresis, or slab-gel electrophoresis.

The methods of the invention may further include the step of obtaining the sample from the subject prior to comparing the level of tumor-specific GM-CSF in the sample to the reference GM-CSF level. Obtaining the sample from the subject and comparing GM-CSF levels can be carried out by the same party or different parties.

In the methods and kits of the invention, potentially any cancer may be treated depending upon the type of cancer vaccine utilized. In some embodiments, the cancer is a B-cell malignancy. In some embodiments, the B-cell malignancy is selected from the group consisting of non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, Burkitt lymphoma/leukemia. In some embodiments, the B-cell malignancy is a mature B-cell lymphoma. In some embodiments, the B-cell malignancy is a mature B-cell lymphoma selected from the group consisting of B-cell chronic lymphocytic leukemia/small lymphocytic lymphoma, B-cell prolymphocytic leukemia, lymphoplasmacytic lymphoma, splenic marginal zone B-cell lymphoma (½ villous lymphocytes), hairy cell leukemia, plasma cell myeloma/plasmacytoma, extranodal marginal zone B-cell lymphoma of MALT type, nodal marginal zone B-cell lymphoma (½ monocytoid B cells), follicular lymphoma, mantle-cell lymphoma, diffuse large B-cell lymphoma, mediastinal large B-cell lymphoma, primary effusion lymphoma, Burkitt lymphoma/Burkitt cell leukemia. In some embodiments, the B-cell malignancy is mantle cell lymphoma or follicular lymphoma. In some embodiments, the cancer is a B-cell malignany, such as mantle cell lymphoma, follicular lymphoma, or another B-cell malignancy, and the cancer vaccine is an autologous idiotype vaccine (e.g., a hybridoma-derived autologous idiotype vaccine).

In the methods of the invention, various cancer vaccines may potentially be utilized to treat the cancer. In some embodiments, the cancer vaccine is an autologous idiotype vaccine, such as a hybridoma-derived idiotype vaccine or a recombinant idiotype vaccine. In some embodiments, the cancer vaccine is selected from among a peptide vaccine, plasmid DNA vaccine, recombinant viral vector, recombinant bacteria, dendritic cell vaccine, tumor cell vaccine, heat-shock protein, or exosome-based vaccine.

In the methods of the invention, the cancer treatment may further comprise an additional (different) cancer treatment. For example, the additional cancer treatment may include chemotherapy, radiation, immunotherapy, or a combination of two or more of the foregoing.

In some embodiments of the methods of the invention, the subject has previously undergone a different therapy for treatment of the cancer. In some embodiments, the different therapy comprises chemotherapy and/or immunotherapy. In some embodiments, the different therapy comprises administration of a monoclonal antibody (e.g., anti-CD20 antibody, such as rituximab). In some embodiments, the different therapy comprises a radioimmunotherapy. In some embodiments, the different therapy comprises a regimen of PACE (prednisone, doxorubicin, cyclophosphamide, and etoposide), CHOP (cyclophosphamide, doxorubicin, vincristine, and prednisone), CHOP-R (cyclophosphamide, doxorubicin, vincristine, prednisone, rituximab), B-R (bendamustine and rituximab), CVP (cyclophosphamide, vincristine, and prednisone), CVP-R (cyclophosphamide, vincristine, prednisone, and rituximab), F-R (fluradarabine and rituximab), FND-R (fludarabine, mitoxantrone, dexamethasone, and rituximab), FCM (fludarabine, cyclophosphamide, and mitoxantrone), FCM-R (fludarabine, cyclophosphamide, mitoxantrone, and rituximab), radioimmunotherapy, single agent rituximab, single agent alkylator, lenalidomide, involved field radiation therapy, or stem cell transplant.

In some embodiments, the subject has undergone an immunochemotherapy induction regimen for treatment of the cancer, such as a regimen comprising administration of a monoclonal antibody directed against CD20 antigen, and chemotherapy. In some embodiments, the monoclonal antibody is rituximab. In some embodiments, the immunochemotherapy induction regimen is a rituximab-containing combination chemotherapy comprises or consists of etoposide, vincristine, doxorubicin, cyclophosphamide, prednisone, and ritruximab (EPOCH-R).

In some embodiments, the subject is in complete remission at the time the cancer vaccine is administered to the subject. In some embodiments, the subject is not in complete remission at the time the cancer vaccine is administered to the subject.

In the methods of the invention, the subject may be human or a non-human animal, such as an animal model.

### DEFINITIONS

In order that the present disclosure may be more readily understood, certain terms are first defined. Additional definitions are set forth throughout the detailed description.

The term “cancer vaccine” refers to prophylactic cancer vaccines and/or therapeutic cancer vaccines such as those that mediate their therapeutic effect through in vivo induction or amplification of the antigen-specific host immune response (e.g., induction of an antigen-specific T-cell response or amplifying a pre-existing antigen-specific T-cell response, especially cytotoxic T-cell responses). The term is inclusive of various cancer vaccine modalities and platforms including but not limited to cellular-based vaccines (e.g., whole tumor cells, gene-modified tumor cells, dendritic cells), protein-based vaccines (e.g., proteins, peptides, agonist peptides, anti-idiotype mAb, mAb fusion proteins), and vector-based vaccines (e.g., viral vectors, bacterial vectors, yeast vectors, plasmid DNA). In some embodiments, the cancer vaccine is an autologous idiotype vaccine, such as a hybridoma-derived idiotype vaccine or a recombinant idiotype vaccine.

The terms “eliminating,” “substantially reducing,” “treating,” and “treatment,” as used herein, refer to therapeutic or preventative measures described herein. The methods of “eliminating or substantially reducing” employ administration to a subject having a cancer, such a B-cell malignancy or other cancer. In some embodiments, the term “eliminating” refers to a complete remission of cancer in a subject treated using the methods described herein. In some embodiments, a subject is in complete remission at the time the cancer vaccine is administered, which may be achieved, for example, through a chemotherapeutic and/or immunotherapeutic induction regimen.

The terms “B lymphocyte” and “B cell,” as used interchangeably herein, are intended to refer to any cell within the B cell lineage as early as B cell precursors, such as pre-B cells B220⁺ cells which have begun to rearrange Ig VH genes and up to mature B cells and even plasma cells such as, for example, plasma cells which are associated with multiple myeloma. The term “B-cell,” also includes a B-cell derived cancer stem cell, i.e., a stem cell which is capable of giving rise to non-Hodgkin's lymphoma, Hodgkin's lymphoma, chronic lymphocytic leukemia, mantle cell lymphoma or multiple myeloma. Such cells can be readily identified by one of ordinary skill in the art using standard techniques known in the art and those described herein.

The terms “cancer” and “malignancy” are used herein interchangeably to refer to or describe the physiological condition in mammals that is typically characterized by unregulated cell growth. The cancer may be a drug-resistant or drug-sensitive type. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, bladder cancer, colorectal cancer, endometrial carcinoma, kidney cancer, and thyroid cancer. In some embodiments, the cancer is mantle cell lymphoma, follicular lymphoma, or another B-cell malignancy.

Other non-limiting examples of cancers are basal cell carcinoma, biliary tract cancer; bone cancer; brain and CNS cancer; choriocarcinoma; connective tissue cancer; esophageal cancer; eye cancer; cancer of the head and neck; gastric cancer; intra-epithelial neoplasm; larynx cancer; lymphoma including Hodgkin's and Non-Hodgkin's lymphoma; melanoma; myeloma; neuroblastoma; oral cavity cancer (e.g., lip, tongue, mouth, and pharynx); retinoblastoma; rhabdomyosarcoma; rectal cancer; cancer of the respiratory system; sarcoma; skin cancer; stomach cancer; testicular cancer; uterine cancer; cancer of the urinary system, as well as other carcinomas and sarcomas. Examples of cancer types that may potentially be prognosticated and/or treated using the kits and methods of the present invention are also listed in Table 1.

TABLE 1
Examples of Cancer Types

| | |
|---|---|
| Acute Lymphoblastic Leukemia, Adult | Hairy Cell Leukemia |
| Acute Lymphoblastic Leukemia, | Head and Neck Cancer |
| Childhood | Hepatocellular (Liver) Cancer, Adult (Primary) |
| Acute Myeloid Leukemia, Adult | Hepatocellular (Liver) Cancer, Childhood |
| Acute Myeloid Leukemia, Childhood | (Primary) |
| Adrenocortical Carcinoma | Hodgkin's Lymphoma, Adult |
| Adrenocortical Carcinoma, Childhood | Hodgkin's Lymphoma, Childhood |
| AIDS-Related Cancers | Hodgkin's Lymphoma During Pregnancy |
| AIDS-Related Lymphoma | Hypopharyngeal Cancer |
| Anal Cancer | Hypothalamic and Visual Pathway Glioma, |
| Astrocytoma, Childhood Cerebellar | Childhood |
| Astrocytoma, Childhood Cerebral | Intraocular Melanoma |
| Basal Cell Carcinoma | Islet Cell Carcinoma (Endocrine Pancreas) |
| Bile Duct Cancer, Extrahepatic | Kaposi's Sarcoma |
| Bladder Cancer | Kidney (Renal Cell) Cancer |
| Bladder Cancer, Childhood | Kidney Cancer, Childhood |
| Bone Cancer, Osteosarcoma/Malignant | Laryngeal Cancer |
| Fibrous Histiocytoma | Laryngeal Cancer, Childhood |
| Brain Stem Glioma, Childhood | Leukemia, Acute Lymphoblastic, Adult |
| Brain Tumor, Adult | Leukemia, Acute Lymphoblastic, Childhood |
| Brain Tumor, Brain Stem Glioma, | Leukemia, Acute Myeloid, Adult |
| Childhood | Leukemia, Acute Myeloid, Childhood |
| Brain Tumor, Cerebellar Astrocytoma, | Leukemia, Chronic Lymphocytic |
| Childhood | Leukemia, Chronic Myelogenous |
| Brain Tumor, Cerebral | Leukemia, Hairy Cell |
| Astrocytoma/Malignant Glioma, | Lip and Oral Cavity Cancer |
| Childhood | Liver Cancer, Adult (Primary) |
| Brain Tumor, Ependymoma, Childhood | Liver Cancer, Childhood (Primary) |
| Brain Tumor, Medulloblastoma, | Lung Cancer, Non-Small Cell |
| Childhood | Lung Cancer, Small Cell |
| Brain Tumor, Supratentorial Primitive | Lymphoma, AIDS-Related |
| Neuroectodermal Tumors, Childhood | Lymphoma, Burkitt's |
| Brain Tumor, Visual Pathway and | Lymphoma, Cutaneous T-Cell, see Mycosis |
| Hypothalamic Glioma, Childhood | Fungoides and Sézary Syndrome |
| Brain Tumor, Childhood | Lymphoma, Hodgkin's, Adult |
| Breast Cancer | Lymphoma, Hodgkin's, Childhood |
| Breast Cancer, Childhood | Lymphoma, Hodgkin's During Pregnancy |
| Breast Cancer, Male | Lymphoma, Non-Hodgkin's, Adult |
| Bronchial Adenomas/Carcinoids, | Lymphoma, Non-Hodgkin's, Childhood |
| Childhood | Lymphoma, Non-Hodgkin's During Pregnancy |
| Burkitt's Lymphoma | Lymphoma, Primary Central Nervous System |
| Carcinoid Tumor, Childhood | Macroglobulinemia, Waldenström's |
| Carcinoid Tumor, Gastrointestinal | Malignant Fibrous Histiocytoma of |
| Carcinoma of Unknown Primary | Bone/Osteosarcoma |
| Central Nervous System Lymphoma, | Medulloblastoma, Childhood |
| Primary | Melanoma |
| Cerebellar Astrocytoma, Childhood | Melanoma, Intraocular (Eye) |
| Cerebral Astrocytoma/Malignant Glioma, | Merkel Cell Carcinoma |
| Childhood | Mesothelioma, Adult Malignant |
| Cervical Cancer | Mesothelioma, Childhood |
| Childhood Cancers | Metastatic Squamous Neck Cancer with Occult |
| Chronic Lymphocytic Leukemia | Primary |
| Chronic Myelogenous Leukemia | Multiple Endocrine Neoplasia Syndrome, |
| Chronic Myeloproliferative Disorders | Childhood |
| Colon Cancer | Multiple Myeloma/Plasma Cell Neoplasm |
| Colorectal Cancer, Childhood | Mycosis Fungoides |
| Cutaneous T-Cell Lymphoma, see | Myelodysplastic Syndromes |
| Mycosis Fungoides and Sezary | Myelodysplastic/Myeloproliferative Diseases |
| Syndrome | Myelogenous Leukemia, Chronic |
| Endometrial Cancer | Myeloid Leukemia, Adult Acute |
| Ependymoma, Childhood | Myeloid Leukemia, Childhood Acute |
| Esophageal Cancer | Myeloma, Multiple |
| Esophageal Cancer, Childhood | Myeloproliferative Disorders, Chronic |
| Ewing's Family of Tumors | Nasal Cavity and Paranasal Sinus Cancer |
| Extracranial Germ Cell Tumor, | Nasopharyngeal Cancer |
| Childhood | Nasopharyngeal Cancer, Childhood |
| Extragonadal Germ Cell Tumor | Neuroblastoma |
| Extrahepatic Bile Duct Cancer | Non-Hodgkin's Lymphoma, Adult |
| Eye Cancer, Intraocular Melanoma | Non-Hodgkin's Lymphoma, Childhood |
| Eye Cancer, Retinoblastoma | Non-Hodgkin's Lymphoma During Pregnancy |
| Gallbladder Cancer | Non-Small Cell Lung Cancer |
| Gastric (Stomach) Cancer | Oral Cancer, Childhood |
| Gastric (Stomach) Cancer, Childhood | Oral Cavity Cancer, Lip and |
| Gastrointestinal Carcinoid Tumor | Oropharyngeal Cancer |
| Germ Cell Tumor, Extracranial, | Osteosarcoma/Malignant Fibrous Histiocytoma |
| Childhood | of Bone |
| Germ Cell Tumor, Extragonadal | Ovarian Cancer, Childhood |
| Germ Cell Tumor, Ovarian | Ovarian Epithelial Cancer |
| Gestational Trophoblastic Tumor | Ovarian Germ Cell Tumor |
| Glioma, Adult | Ovarian Low Malignant Potential Tumor |
| Glioma, Childhood Brain Stem | Pancreatic Cancer |
| Glioma, Childhood Cerebral | Pancreatic Cancer, Childhood |
| Astrocytoma | Pancreatic Cancer, Islet Cell |
| Glioma, Childhood Visual Pathway and | Paranasal Sinus and Nasal Cavity Cancer |
| Hypothalamic | Parathyroid Cancer |
| Skin Cancer (Melanoma) | Penile Cancer |
| Skin Carcinoma, Merkel Cell | Pheochromocytoma |
| Small Cell Lung Cancer | Pineoblastoma and Supratentorial Primitive |
| Small Intestine Cancer | Neuroectodermal Tumors, Childhood |
| Soft Tissue Sarcoma, Adult | Pituitary Tumor |
| Soft Tissue Sarcoma, Childhood | Plasma Cell Neoplasm/Multiple Myeloma |
| Squamous Cell Carcinoma, see Skin | Pleuropulmonary Blastoma |
| Cancer (non-Melanoma) | Pregnancy and Breast Cancer |
| Squamous Neck Cancer with Occult | Pregnancy and Hodgkin's Lymphoma |
| Primary, Metastatic | Pregnancy and Non-Hodgkin's Lymphoma |
| Stomach (Gastric) Cancer | Primary Central Nervous System Lymphoma |
| Stomach (Gastric) Cancer, Childhood | Prostate Cancer |
| Supratentorial Primitive | Rectal Cancer |
| Neuroectodermal Tumors, Childhood | Renal Cell (Kidney) Cancer |
| T-Cell Lymphoma, Cutaneous, see | Renal Cell (Kidney) Cancer, Childhood |
| Mycosis Fungoides and Sezary | Renal Pelvis and Ureter, Transitional Cell |
| Syndrome | Cancer |
| Testicular Cancer | Retinoblastoma |
| Thymoma, Childhood | Rhabdomyosarcoma, Childhood |
| Thymoma and Thymic Carcinoma | Salivary Gland Cancer |
| Thyroid Cancer | Salivary Gland Cancer, Childhood |
| Thyroid Cancer, Childhood | Sarcoma, Ewing's Family of Tumors |
| Transitional Cell Cancer of the Renal | Sarcoma, Kaposi's |
| Pelvis and Ureter | Sarcoma, Soft Tissue, Adult |
| Trophoblastic Tumor, Gestational | Sarcoma, Soft Tissue, Childhood |
| Unknown Primary Site, Carcinoma of, | Sarcoma, Uterine |
| Adult | Sezary Syndrome |
| Unknown Primary Site, Cancer of, | Skin Cancer (non-Melanoma) |
| Childhood | Skin Cancer, Childhood |
| Unusual Cancers of Childhood | |
| Ureter and Renal Pelvis, Transitional Cell | |
| Cancer | |
| Urethral Cancer | |
| Uterine Cancer, Endometrial | |
| Uterine Sarcoma | |
| Vaginal Cancer | |
| Visual Pathway and Hypothalamic | |
| Glioma, Childhood | |
| Vulvar Cancer | |
| Waldenström's Macroglobulinemia | |
| Wilms' Tumor | |

As used herein, the term “tumor” refers to all neoplastic cell growth and proliferation, whether malignant or benign, and all pre-cancerous and cancerous cells and tissues. For example, a particular cancer may be characterized by a solid mass tumor or non-solid tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography) or magnetic resonance imaging (MRI), or by needle aspirations. The use of these latter techniques is more common in early detection. Molecular and phenotypic analysis of cancer cells within a tissue can usually be used to confirm if the cancer is endogenous to the tissue or if the lesion is due to metastasis from another site. The prognostication and treatment methods of the invention can be utilized for early, middle, or late stage disease, and acute or chronic disease.

The terms “B-cell malignancy” and “B-cell derived malignancy” refer to a malignancy arising from aberrant replication of B cells. B-cell malignancies include, for example, non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, Burkitt lymphoma/leukemia. The B-cell malignancy may be a mature B-cell lymphoma. Examples of mature B-cell lymphomas include B-cell chronic lymphocytic leukemia/small lymphocytic lymphoma, B-cell prolymphocytic leukemia, lymphoplasmacytic lymphoma, splenic marginal zone B-cell lymphoma (½ villous lymphocytes), hairy cell leukemia, plasma cell myeloma/plasmacytoma, extranodal marginal zone B-cell lymphoma of MALT type, nodal marginal zone B-cell lymphoma (½ monocytoid B cells), follicular lymphoma, mantle-cell lymphoma, diffuse large B-cell lymphoma, mediastinal large B-cell lymphoma, primary effusion lymphoma, Burkitt lymphoma/Burkitt cell leukemia.

The mature B-cell lymphoma may be a variant malignancy, for example, B-cell chronic lymphocytic leukemia/small lymphocytic lymphoma with monoclonal gammopathy/plasmacytoid differentiation, hairy cell leukemia variant, cutaneous follicle center lymphoma, diffuse follicle center lymphoma, blastoid mantle-cell lymphoma, morphologic variant of diffuse large B-cell lymphoma (for example, centroblastic, immunoblastic, T-cell/histiocyte-rich, lymphomatoid granulomatosis type, anaplastic large B-cell, plasmablastic) or subtype of diffuse large B-cell lymphoma (for example, mediastinal (thymic) large B-cell lymphoma, primary effusion lymphoma, intravascular large B-cell lymphoma), morphologic variant of Burkitt lymphoma or Burkitt cell leukemia (for example, Burkitt-like lymphoma/leukemia, Burkitt lymphoma/Burkitt cell leukemia with plasmacytoid differentiation (AIDS-associated), or clinical or genetic subtype of Burkitt lymphoma/Burkitt cell leukemia (for example, endemic, sporadic, immunodeficiency-associated).

The terms “immunoglobulin” and “antibody” (used interchangeably herein) include a protein having a basic four-polypeptide chain structure consisting of two heavy and two light chains, said chains being stabilized, for example, by interchain disulfide bonds, which has the ability to specifically bind an antigen. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind an antigen. The term “domain” refers to a globular region of a heavy or light chain polypeptide comprising peptide loops (e.g., comprising 3 to 4 peptide loops) stabilized, for example, by β-pleated sheet and/or intrachain disulfide bond. Domains are further referred to herein as “constant” or “variable,” based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions.” The “constant” domains of an antibody light chain are referred to interchangeably as “light chain constant regions,” “light chain constant domains,” “CL” regions or “CL” domains. The “constant” domains of an antibody heavy chain are referred to interchangeably as “heavy chain constant regions,” “heavy chain constant domains,” “CH” regions or “CH” domains). The “variable” domains of an antibody light chain are referred to interchangeably as “light chain variable regions,” “light chain variable domains,” “VL” regions or “VL” domains). The “variable” domains of an antibody heavy chain are referred to interchangeably as “heavy chain constant regions,” “heavy chain constant domains,” “VH” regions or “VH” domains).

Immunoglobulins or antibodies can exist in monomeric or polymeric form, for example, IgM antibodies which exist in pentameric form and/or IgA antibodies which exist in monomeric, dimeric or multimeric form. Other than “bispecific” or “bifunctional” immunoglobulins or antibodies, an immunoglobulin or antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79:315-321; Kostelny et al., (1992) J. Immunol. 148:1547-1553.

The term “antigen-binding portion” of an antibody (or “antibody portion”) includes fragments of an antibody that retain the ability to specifically bind to an antigen (e.g., a B-cell specific antigen). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P. et al., (1993) Proc. Natl. Acad. Sci. USA 90:6444-6448; Poljak, R. J. et al., (1994) Structure 2:1121-1123). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M. et al., (1995) Human Antibodies and Hybridomas 6:93-101) and use of a cysteine residue, a marker peptide and a C-terminal polyhistidine tag to make bivalent and biotinylated scFv molecules (Kipriyanov, S. M. et al., (1994) Mol. Immunol., 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Preferred antigen binding portions are complete domains or pairs of complete domains.

“Specific binding,” “specifically binds,” “specific for”, “selective binding,” and “selectively binds,” as used herein, mean that the compound, e.g., antibody or antigen-binding portion thereof, exhibits appreciable affinity for a particular antigen or epitope and, generally, does not exhibit significant cross-reactivity with other antigens and epitopes. “Appreciable” or preferred binding includes binding with an affinity of at least 10⁶, 10⁷, 10⁸, 10⁹ M⁻¹, or 10¹⁰ M⁻¹. Affinities greater than 10⁷M⁻¹, preferably greater than 10⁸ M⁻¹ are more preferred. Values intermediate of those set forth herein are also intended to be within the scope of the present invention and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross-reactivity” is one that will not appreciably bind to an undesirable entity (e.g., an undesirable proteinaceous entity). For example, in one embodiment, an antibody or antigen-binding portion thereof, that specifically binds to a B-cell specific antigen, such as, for example, CD-20 or CD-22, will appreciably bind CD-20 or CD-22, but will not significantly react with other non-CD-20 or non-CD-22 proteins or peptides. Specific or selective binding can be determined according to any art-recognized means for determining such binding, including, for example, according to Scatchard analysis and/or competitive binding assays.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The term “human antibody” includes antibodies having variable and constant regions corresponding to human germline immunoglobulin sequences as described by Kabat et al. (See Kabat, et al., (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In a preferred embodiment, these replacements are within the CDR regions as described in detail below.

The term “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor, L. D. et al., (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat E. A., et al., (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or backmutation or both.

An “isolated antibody” includes an antibody that is substantially free of other antibodies having different antigenic specificities (e.g., an isolated antibody that specifically binds a B-cell specific antigen and is substantially free of antibodies or antigen-binding portions thereof that specifically bind other antigens, including other B-cell antigens). An isolated antibody that specifically binds a B-cell specific antigen may bind the same antigen and/or antigen-like molecules from other species. Moreover, an isolated antibody may be substantially free of other cellular material and/or chemicals.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The terms “idiotype,” “Id,” and “idiotypic determinant,” as used herein, refer to an epitope in the hypervariable region of an immunoglobulin. Typically, an idiotype or an epitope thereof is formed by the association of the hypervariable or complementarity determining regions (CDRs) of VH and VL domains.

The terms “anti-idiotype” and “anti-Id,” refer to an antibody, or antigen-binding portion thereof, that binds one or more idiotypes present on an antibody.

The cancer vaccine may be an autologous idiotype vaccine. The term “autologous idiotype vaccine” refers to a composition, the active ingredient of which is an immunogenic molecule that is preferably capable of inducing an immune response against a B-cell idiotype derived from the same subject to which it is administered. In some embodiments, the immunogenic molecule in a vaccine used in the methods of the present invention is a normal product of a subject's B cells that happens to be expressed clonally on the cancer cells (e.g., cells derived from a Hodgkin's lymphoma or non-Hodgkin's lymphoma or chronic lymphocytic leukemia, mantle cell lymphoma or multiple myeloma) and serves as a unique a target for immune attack. In some embodiments, the vaccine comprises an IgM anti-Id immunoglobulin. In some embodiments, an “autologous idiotype vaccine,” is capable of eliciting an immune response against a B-cell idiotype derived from a subject having non-Hodgkin's lymphoma. In another embodiment, an “autologous idiotype vaccine,” is capable of eliciting an immune response against a B-cell idiotype derived from a subject having Hodgkin's lymphoma. In yet another embodiment, an “autologous idiotype vaccine,” is capable of eliciting an immune response against a B-cell idiotype derived from a subject having chronic lymphocytic leukemia. In a further embodiment, an “autologous idiotype vaccine,” is capable of eliciting an immune response against a B-cell idiotype derived from a subject having multiple myeloma. In a yet further embodiment, an “autologous idiotype vaccine,” is capable of eliciting an immune response against a B-cell idiotype derived from a subject having mantle cell lymphoma. In some embodiments of the present invention, an “autologous idiotype vaccine,” is used for the treatment of a B-cell derived cancer in combination with other immune therapeutics such as, for example, monoclonal antibodies that selectively bind B-cell specific antigens. In some embodiments, an “autologous idiotype vaccine” includes an antigen associated with a B-cell derived cancer in a subject (e.g., non-Hodgkin's lymphoma, Hodgkin's lymphoma, chronic lymphocytic leukemia, mantle cell lymphoma or multiple myeloma) linked to KLH (keyhole limpet hemocyanin, a carrier protein). In some embodiments of the present invention, an autologous idiotype vaccine is administered in conjunction with GM-CSF, and subsequently re-administered, as a booster, one or times with or without GM-CSF.

The term “granulocyte monocyte colony stimulating factor” or “GM-CSF” refers to a hematopoeitic growth factor that stimulates the development of committed progenitor cells to neutrophils and enhances the functional activities of neutrophils (GM-CSF (GenBank accession number M11220.1 version GI:183363; Lee F. et al., Proc. Natl. Acad. Sci USA, 1985 July; 82(13):4360-4364). It is produced in response to specific stimulation by a variety of cells including macrophages, fibroblasts, endothelial cells and bone marrow stroma. Either purified GM-CSF or recombinant GM-CSF, for example, recombinant human GM-CSF (R & D SYSTEMS, INC, Minneapolis, Minn.) or sargramostim (LEUKINE, BAYER HEALTHCARE Pharmaceuticals, Wayne, N.J.) can be used for exogenous GM-CSF in the methods described herein.

In some embodiments, GM-CSF is administered to the subject, in addition to the cancer vaccine. The phrase “an effective amount of granulocyte monocyte colony stimulating factor” refers to an amount of granulocyte monocyte colony stimulating factor (GM-CSF), which upon a single or multiple dose administration to a subject, induces or enhances an immune response in the subject (e.g., as an adjuvant). In some embodiments, 50 μg/m²/day to about 200 μg/m²/day (e.g., 100 μg/m²/day) granulocyte monocyte colony stimulating factor is administered to the subject. In some embodiments, “an effective amount of granulocyte monocyte colony stimulating factor” refers to a daily administration of 5 μg/kg of the granulocyte colony stimulating factor.

As used herein, the term “antigen” refers to a molecule (for example, a polypeptide, nucleic acid molecule, carbohydrate, glycoprotein, lipid, lipoprotein, glycolipid, or small molecule) that is capable of eliciting an immune response and contains an epitope or antigenic determinant to which an immunoglobulin can specifically bind.

As used herein, the term “epitope” or “antigenic determinant” or “idiotypic determinant” refers to a site on an antigen to which an immunoglobulin (or antigen binding fragment thereof) can specifically bind. Epitopes can be formed both from contiguous amino acids or noncontiguous amino acids juxtaposed by tertiary folding of a protein. Epitopes found on the Fab (variable) region of immunoglobulins are referred to as “idiotypic determinants” and comprise the immunoglobulin's “idiotype”. Epitopes formed from contiguous amino acids are typically retained on exposure to denaturing solvents, whereas epitopes formed by tertiary folding are typically lost on treatment with denaturing solvents. In the case of proteinaceous antigens, an epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, for example, Epitope Mapping Protocols in Methods in Molecular Biology, Vol. 66, G. E. Morris, Ed. (1996).

The term “domain” refers to a globular region of a heavy or light chain polypeptide comprising peptide loops (e.g., comprising 3 to 4 peptide loops) stabilized, for example, by beta-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. “Constant” domains on the light chain are referred to interchangeably as “light chain constant regions”, “light chain constant domains”, “CL” regions or “CL” domains). “Constant” domains on the heavy chain are referred to interchangeably as “heavy chain constant regions”, “heavy chain constant domains”, “CH” regions or “CH” domains). “Variable” domains on the light chain are referred to interchangeably as “light chain variable regions”, “light chain variable domains”, “VL” regions or “VL” domains). “Variable” domains on the heavy chain are referred to interchangeably as “heavy chain variable regions”, “heavy chain variable domains”, “VH” regions or “VH” domains).

The term “region” refers to a part or portion of an antibody chain or antibody chain domain (for example, a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein. As used herein, a “region” of an antibody is inclusive of regions existing in isolation (as antibody fragments) and as part of whole (intact) or complete antibodies. Thus, for example, an idiotype immunoglobulin comprising “at least an IgM constant region” encompasses embodiments in which the idiotype immunoglobulin is composed of only the constant region of the IgM (and, optionally, other non-IgM components), as well as embodiments in which the idiotype immunoglobulin is composed of more of the IgM than just the constant region (and, optionally, other non-IgM components).

As used herein, the terms “constant region” or “fragment crystallizable region” (Fc region) refers to that portion of the antibody (the tail region) that interacts with cell surface receptors called Fc receptors and some proteins of the complement system, and is composed of two heavy chains that contribute two or three constant domains depending on the class of the antibody (Janeway C A, Jr et al. (2001) Immunobiology. (5th ed.); Garland Publishing). In IgG, IgA and IgD antibody isotypes, the Fc region is composed of two identical protein fragments, derived from the second and third constant domains of the antibody's two heavy chains; IgM and IgE Fe regions contain three heavy chain constant domains (C_{H} domains 2-4) in each polypeptide chain. The Fc regions of IgGs bear a highly conserved N-glycosylation site (Janeway Calif., Jr et al. (2001) Immunobiology. (5th ed.); Garland Publishing Rhoades RA, Pflanzer RG (2002) Human Physiology (4th ed.); Thomson Learning). The other part of an antibody, called the Fab region, contains variable sections that define the specific target that the antibody can bind. By contrast, the Fe region of all antibodies in a class are the same for each species; they are constant rather than variable. The terms “Fc region” and “Fab region” encompass these regions existing in isolation (as antibody fragments) and as part of a whole (intact) or complete, full-length antibody.

The cancer vaccine may be a gene expression vaccine, comprising a polynucleotide encoding a polypeptide against which an immune response is desired. The terms “polynucleotide” and “nucleic acid molecule” are used interchangeably herein to refer to a polymeric form of nucleotides of any length, which contain deoxyribonucleotides, ribonucleotides, and analogs in any combination analogs. Polynucleotides may have any three-dimensional structure, and may perform any function, known or unknown. The term “nucleic acid molecule” includes double-, single-stranded, and triple-helical molecules. Unless otherwise specified or required, any embodiment of the invention described herein that is a nucleic acid molecule encompasses both the double-stranded form and each of two complementary single-stranded forms known or predicted to make up the double stranded form. In some embodiments, the nucleic acid molecule encodes an epitope or an antigen.

The following are non-limiting examples of nucleic acid molecules: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A nucleic acid molecule may be further modified after polymerization, such as by conjugation with a labeling component. Other types of modifications included in this definition are caps, substitution of one or more of the naturally occurring nucleotides with an analog, and introduction of means for attaching to proteins, metal ions, labeling components, other nucleic acid molecules, or a solid support.

The cancer vaccine may comprise a polypeptide (such as tumor-specific antigen or tumor-associated antigen). The terms “polypeptide”, “peptide” and “protein” are used interchangeably herein to refer to polymers of amino acids of any length. The polymer may be linear or branched, it may comprise modified amino acids or amino acid analogs, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component.

The cancer vaccine may be a fusion polypeptide. The term “fusion polypeptide” refers to a polypeptide comprising regions in a different position in the sequence than occurs in nature. The regions may normally exist in separate proteins and are brought together in the fusion polypeptide; or they may normally exist in the same protein but are pieced in a new arrangement in the fusion polypeptide. Fusion polypeptides can be produced by linking two or more polypeptides together (for example, covalently), or by expressing nucleic acids encoding each fusion partner within a host cell, for example. The cancer vaccine may be a fusion polypeptide (e.g., a TAA or TSA directly or indirectly linked to a heterologous polypeptide, such as an adjuvant).

The cancer vaccine may be administered with or without an adjuvant. The term “adjuvant” refers to a substance incorporated into or administered simultaneously with an antigen which potentiates the immune response in response to that antigen but does not in itself confer immunity. A tetanus, diphtheria, and pertussis vaccine, for example, contains minute quantities of toxins produced by each of the target bacteria, but also contains some aluminum hydroxide. Aluminum salts are common adjuvants in vaccines sold in the United States and have been used in vaccines for over 70 years. The body's immune system develops an antitoxin to the bacteria's toxins, not to the aluminum, but would not respond enough without the help of the aluminum adjuvant. An adjuvant can also include cytokines such as granulocyte-monocyte colony stimulating factor (GM-CSF). In some cases, e.g., immunization of a subject against normally non-immunogenic tumor-derived idiotypes, foreign (non-self) carrier protein immunogens such as keyhole limpet hemocyanin (KLH), can also potentiate the immune response and serve as adjuvants.

As used herein, the terms “treat” or “treatment” refer to both therapeutic treatment and prophylactic or preventative measures, wherein the object is to prevent or slow down (lessen) an undesired physiological change or disorder, such as the development or spread of cancer or other disorder. For purposes of this invention, beneficial or desired clinical results (i.e., a positive clinical outcome) include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. For example, treatment with a cancer vaccine in accordance with the invention can result in therapeutic treatment or prophylaxis of cancer. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject may be identified (e.g., diagnosed) as one suffering from the disease or condition prior to treatment with the cancer vaccine.

As used herein, the term “(therapeutically) effective amount” refers to an amount of a cancer vaccine effective to a cancer in a mammalian subject (human or non-human mammal). In the case of cancer (which includes pre-cancer), the therapeutically effective amount may reduce (i.e., slow to some extent and preferably stop) unwanted cellular proliferation; reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer (i.e., a positive clinical outcome). To the extent administration prevents growth of and/or kills existing cancer cells, it may be cytostatic and/or cytotoxic. For cancer therapy, efficacy or effectiveness of a cancer vaccine can, for example, be measured by assessing the time to disease progression (TTP), determining the response rate (RR), overall survival (OS), time-to-next-treatment (TNTT), or time from first progression to next treatment, or a combination of two or more of the foregoing. The amount of cancer vaccine may be a growth inhibitory amount. Preferably, the amount of cancer vaccine is an amount determined to induce a level of tumor-specific GM-CSF that is consistent with a positive clinical outcome such as the aforementioned outcomes in a similar demographic population having a cancer of the same type.

As used herein, the term “growth inhibitory amount” refers to an amount which inhibits growth or proliferation of a target cell, such as a tumor cell, either in vitro or in vivo, irrespective of the mechanism by which cell growth is inhibited (e.g., by cytostatic properties, cytotoxic properties, etc.). In a preferred embodiment, the growth inhibitory amount inhibits (i.e., slows to some extent and preferably stops) proliferation or growth of the target cell in vivo or in cell culture by greater than about 20%, preferably greater than about 50%, most preferably greater than about 75% (e.g., from about 75% to about 100%).

As used herein, the term “positive clinical outcome” will vary with the disease. For example, depending upon the type of cancer, a positive clinical outcome may be selected from alleviation of one or more symptoms of the cancer, diminishment of extent of disease, stabilized (i.e., not worsening) state of disease, delay or slowing of disease progression, amelioration or palliation of the disease state, remission (whether partial or total), whether detectable or undetectable, tumor regression, inhibition of tumor growth, inhibition of tumor metastasis, reduction in cancer cell number, inhibition of cancer cell infiltration into peripheral organs, improved time to disease progression (TTP), improved response rate (RR), prolonged overall survival (OS), prolonged time-to-next-treatment (TNTT), or prolonged time from first progression to next treatment, or a combination of two or more of the foregoing.

As used herein, the terms “patient”, “subject”, and “individual” are used interchangeably and are intended to include human and non-human animal species. For example, the subject may be a human or an animal model.

As used in this specification, the singular forms “a”, “an”, and “the” include plural reference unless the context clearly dictates otherwise. Thus, for example, a reference to “an antibody” means one or more such antibody. A reference to “a molecule” means one or more such molecule, and so forth.

### Assessment of Tumor-Specific GM-CSF Response

The methods of the invention may comprise, after administering the cancer vaccine to the subject, verifying whether the subject has developed a tumor-specific GM-CSF response. The methods of the invention may comprise assessing whether the GM-CSF response has been elicited in the subject and, optionally, determining whether the GM-CSF response has subsequently increased, diminished, or remained the same (e.g., in character and/or extent).

An assessment can be made of the nature and/or extent of the subject's tumor-specific GM-CSF response one or more times after the initial treatment. Preferably, an assessment of the subject's tumor-specific GM-CSF response is also made before the subject's initial treatment with the cancer vaccine (e.g., to establish a control or base-line for comparison to a subsequent assessment or assessments post-treatment). For example, an assessment of GM-CSF response can be made from a sample obtained from the subject before treatment with the cancer vaccine but after treatment with chemotherapy, immunotherapy, or both (immunochemotherapy), such as an immunochemotherapy induction regimen (for example, EPOCH-R).

In the methods and kits of the invention, the subject's tumor-specific GM-CSF response can be monitored by making multiple assessments after the initial treatment at uniform time intervals (e.g., every three months, every six months, every nine months, or annually) or at non-uniform time intervals. Monitoring of the subject's tumor-specific GM-CSF response can continue for a pre-determined period of time, for a time determined based on therapeutic outcome, or indefinitely. Preferably, the subject's tumor-specific GM-CSF response is monitored from a time period starting prior to initial vaccination and continuing for a period of time afterward (for example, for a period of at least five years), or indefinitely through the subject's life.

Typically, each assessment will involve obtaining an appropriate biological sample from the subject. The appropriate biological sample will depend upon the particular aspect of the subject's immune response to be assessed (e.g., depending upon the particular assay). For example, in some embodiments, the biological sample will be one or more specimens selected from among blood, peripheral blood mononuclear cells (PBMC), and a tumor. Samples for assessments are taken at a time point appropriate to obtain information regarding the immune response at the time of interest. For example, a sample may be taken from the subject from a time prior to administration of the epitope and additional samples may be taken from the subject periodically after administration to determine the nature and extent of the immune responses observed. In some embodiments, the sample comprises or consists of helper T-cells or supernatant from a culture of the helper T-cells.

The level of tumor-specific GM-CSF can be determined by determining the level of tumor-specific GM-CSF mRNA or tumor-specific GM-CSF protein. Known immunological monitoring methods may be utilized to determine the level of tumor-specific GM-CSF (see, for example, Whelan M. et al., “Biomarkers For Development of Cancer Vaccines,” Personalized Medicine, 2006, 3(1):79-88, which is incorporated herein by reference in its entirety). The level of tumor-specific GM-CSF can determined using an immunoassay, such as a competitive or immunometric assay. The assay may be, for example, a radioimmunoassay (RIA), immunoradiometric assay (IRMA), enzyme-linked immunosorbent assay (ELISA), or enzyme-linked immunosorbent spot (ELISPOT) assay. The level of tumor-specific GM-CSF can be determined using surface plasmon resonance, fluorescence resonance energy transfer, bioluminescence resonance energy transfer, fluorescence quenching flurorescence, fluorescence polarization, mass spectrometry (MS), high-performance liquid chromatography (HPLC), high-performance liquid chromatography/mass spectrometry (HPLC/MS), high-performance liquid chromatography/mass spectrometry/mass spectrometry (HPLC/MS/MS), capillary electrophoresis, rod-gel electrophoresis, or slab-gel electrophoresis.

Assay standardization can include specific parameters to control for general variability in an immune response, such as assay conditions, sensitivity and specificity of the assay, any in vitro amplification step involved, positive and negative controls, cutoff values for determining positive and negative test results from subjects' samples, and any statistical analytical methods to be used for test results can be determined and selected by one of ordinary skill in the art. For example, a reference level of GM-CSF that the tumor-specific GM-CSF level of the sample is compared against may be, for example, a level from a sample obtained from the subject at an earlier time point (before or after administration of the cancer vaccine), or the reference level of GM-CSF may be a statistically calculated level from an appropriate subject population, representing a level that is consistent with a positive (desired) clinical outcome or that is inconsistent with a positive clinical outcome. The reference level may be a single value (e.g., a cutoff value), a range, etc. For example, the reference level may be a range such that if the subject's tumor-specific GM-CSF level reaches or falls within the range, the subject's GM-CSF level is deemed acceptable and no action need be taken. Conversely, if the subject's tumor-specific GM-CSF level does not reach the reference level or falls outside the acceptable range, this can be indicative that some action should be taken. For example, the frequency and/or dosage of the cancer vaccine may be increased. Alternatively, a different treatment can be utilized (e.g., a different cancer vaccine, different adjuvant, or a different therapeutic agent).

Optionally, additional immune responses are assessed by conducting one or more humoral response assays and/or cellular response assays, such as those described by Neelapu et al. (Nature Medicine, 11(9):986-991 (2005)), which is incorporated herein by reference in its entirety. Peripheral blood B and T cells can be collected from the subject and blood counts can be determined, including but not limited to CD3-CD19+ B cells, CD3+CD4+ T cells, and CD3+CD8+ T cells. Tumor cells can be determined, and PBMCs isolated. Both B-cells and tumor cells can be activated with recombinant CD40 ligand trimer, as described in Neelapu et al. (2005). Depending on the type of immune response to be assessed (e.g., humoral, cellular, or both), one or more of the following assays may be used:
Humoral immune response assay: to assess anti-Id humoral responses and tumor-specific antibodies (see, for example, Kwak et al., Lancet, 345:1016-1020 (1995), which is incorporated herein by reference in its entirety).
IFN-gamma ELISPOT assay: to assess tumor-reactive T-cell precursor frequencies via an IFN-gamma response (see, for example, Malyguine et al., J. Trans. Med., 2:9 (2004) and Neelapu et al., Clin. Cancer Res., 10:8309-8317 (2004), which are each incorporated herein by reference in its entirety).
Cytokine induction assay: to assess biomarkers in the tumor that correlate with clinical outcome following autologous anti-idiotype vaccine therapy (see, for example, Neelapu et al. (2004)).
Intracellular cytokine assay: to assess tumor-specific CD4+ and CD8+ T-cell responses (Neealapu et al., J. Cancer Res. Clin. Oncol., 127 Suppl. 2, R14-19 (2001)).

### Modes of Administration

The cancer vaccine used in the kits and methods described herein may be administered by any route effective for delivery to the desired tissues, e.g., administered orally, parenterally (e.g., intravenously), intramuscularly, sublingually, buccally, rectally, intranasally, intrabronchially, intrapulmonarily, intraperitoneally, topically, transdermally and subcutaneously, for example. The amount administered in a single dose may be dependent on the subject being treated, the subject's weight, the manner of administration and the judgment of the prescribing physician. Generally, however, administration and dosage and the duration of time for which a composition is administered will approximate that which are necessary to achieve a desired result. Optionally, various vaccine delivery systems may be utilized (see, for example, Bolhassani A. et al., “Improvement of Different Vaccine Delivery Systems for Cancer Therapy,” Molecular Cancer, 2011, 10:3, which is incorporated herein by reference in its entirety).

Single or multiple administrations of the cancer vaccine can be carried out with dose levels and pattern being selected by the treating physician, preferably based on the level of tumor-specific GM-CSF in samples obtained from the subject. In any event, the compositions should comprise a quantity of vaccine sufficient to treat the subject as desired.

In general, a therapeutically effective amount of a monoclonal antibody such as, for example, an antibody that specifically binds CD-20 or CD-22, can be from about 0.0001 mg/Kg to 0.001 mg/Kg; 0.001 mg/kg to about 10 mg/kg body weight or from about 0.02 mg/kg to about 5 mg/kg body weight. In some embodiments, a therapeutically effective amount of a monoclonal antibody is from about 0.001 mg to about 0.01 mg, about 0.01 mg to about 100 mg, or from about 100 mg to about 1000 mg, for example.

In some embodiments, a therapeutically effective amount of an autologous idiotype vaccine is from about 0.001 mg to about 0.01 mg, about 0.01 mg to about 100 mg, or from about 100 mg to about 1000 mg, for example. In some embodiments, an effective amount of the autologous idiotype vaccine is one or more doses of 0.5 mg.

In some embodiments, an effective amount of an antibody administered to a subject having non-Hodgkin's lymphoma, Hodgkin's lymphoma, chronic lymphocytic leukemia or multiple myeloma is between about 100 mg/m² and 200 mg/m², or between about 200 mg/m² and 300 mg/m² or between about 300 mg/m² and 400 mg/m². In a particular embodiment, an effective amount of a monoclonal antibody that selectively binds a B-cell specific antigen is about 375 mg/m².

The optimal pharmaceutical formulations can be readily determined by one or ordinary skilled in the art depending upon the route of administration and desired dosage. (See, for example, Remington's Pharmaceutical Sciences, 18th Ed. (1990), Mack Publishing Co., Easton, Pa., the entire disclosure of which is hereby incorporated by reference).

The cancer vaccines can be formulated for the most effective route of administration, including for example, oral, transdermal, sublingual, buccal, parenteral, rectal, intranasal, intrabronchial or intrapulmonary administration.

In some embodiments, the cancer vaccine is administered with one or more cytokines such as, for example, GM-CSF, or other immunostimulatory agents. GM-CSF is a potent immunostimulatory cytokine with efficacy in promoting anti-tumor response, particularly T cell responses. In general, however, any cytokine or chemokine that induces inflammatory responses, recruits antigen presenting cells (APC) to the tumor and, possibly, promotes targeting of antigen presenting cells (APC) may be used, for example.

The cancer vaccines useful in the methods of the present invention may be administered by any conventional route including oral and parenteral. Examples of parenteral routes are subcutaneous, intradermal, transcutaneous, intravenous, intramuscular, intraorbital, intracapsular, intrathecal, intraspinal, intracisternal, intraperitoneal, etc. If booster doses are utilized, the primary treatment and one or more booster doses are preferably administered by the same route, e.g., subcutaneously.

The cancer vaccine and any adjuvant (if administered) can be administered within the same formulation or different formulations. If administered in different formulations, the cancer vaccine and the adjuvant can be administered by the same route or by different routes. Administration is preferably by injection on one or multiple occasions to produce systemic immunity. In general, multiple administrations in a standard immunization protocol are used, as is standard in the art. For example, the vaccines can be administered at approximately two to six week intervals, or monthly, for a period of from one to six inoculations in order to provide protection. Again, the vaccine may be administered by any conventional route including oral and parenteral. Examples of parenteral routes are subcutaneous, intradermal, transcutaneous, intravenous, intramuscular, intraorbital, intracapsular, intrathecal, intraspinal, intracisternal, intraperitoneal, etc.

As indicated above, the cancer vaccine used in the kits and methods of the invention may further include one or more adjuvants or immunostimulatory agents. Examples of adjuvants and immunostimulatory agents include, but are not limited to, aluminum hydroxide, aluminum phosphate, aluminum potassium sulfate (alum), beryllium sulfate, silica, kaolin, carbon, water-in-oil emulsions, oil-in-water emulsions, muramyl dipeptide, bacterial endotoxin, lipid X, whole organisms or subcellular fractions of the bacteria Propionobacterium acnes or Bordetella pertussis, polyribonucleotides, sodium alginate, lanolin, lysolecithin, vitamin A, saponin and saponin derivatives, liposomes, levamisole, DEAE-dextran, blocked copolymers or other synthetic adjuvants. Such adjuvants are readily commercially available.

Depending on the intended mode of administration, the compounds used in the methods and kits described herein may be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. Each dose may include an effective amount of a compound used in the methods described herein in combination with a pharmaceutically acceptable carrier and, in addition, may include other medicinal agents, pharmaceutical agents, carriers, adjuvants, diluents, etc.

Liquid pharmaceutically administrable compositions can prepared, for example, by dissolving, dispersing, etc., a compound for use in the methods described herein and optional pharmaceutical adjuvants in an excipient, such as, for example, water, saline aqueous dextrose, glycerol, ethanol, and the like, to thereby form a solution or suspension. For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; see, for example, Remington's Pharmaceutical Sciences, 18th Ed. (1990), Mack Publishing Co., Easton, Pa., the entire disclosure of which is hereby incorporated by reference).

Formulations comprising cancer vaccines and adjuvants may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. Eds. (1991) IRL Press)), each of which are incorporated herein by reference in their entirety.

Experimental controls are considered fundamental in experiments designed in accordance with the scientific method. It is routine in the art to use experimental controls in scientific experiments to prevent factors other than those being studied from affecting the outcome.

### EXEMPLIFIED EMBODIMENTS

Following are exemplified embodiments of the invention.

### Embodiment 1

A method of prognosticating an outcome of cancer treatment with a cancer vaccine in a subject, comprising comparing the level of tumor-specific (anti-tumor) granulocyte-macrophage colony-stimulating factor (GM-CSF) in a sample obtained from the subject with a reference level of GM-CSF, wherein the level of tumor-specific GM-CSF in the sample compared to the reference level of GM-CSF is prognostic for an outcome of treatment with the cancer vaccine.

### Embodiment 2

A method for treating cancer in a subject with a cancer vaccine, the method comprising: assessing the level of tumor-specific (anti-tumor) granulocyte-macrophage colony-stimulating factor (GM-CSF) in a sample obtained from a subject that has been administered a cancer vaccine for treatment of a cancer; and determining whether the level of tumor-specific GM-CSF has diminished in the subject.

### Embodiment 3

A method for comparing the efficacy of two or more cancer vaccine treatments, comprising comparing the level of tumor-specific (anti-tumor) granulocyte macrophage colony-stimulating factor (GM-CSF) response from a first subject that has received a first cancer vaccine treatment to the level of GM-CSF response from a second subject that has received a second cancer vaccine treatment, wherein the first vaccine cancer treatment and the second cancer vaccine treatment differ in cancer vaccine composition and/or in cancer vaccine administration.

### Embodiment 4

The method of embodiment 3, wherein the first cancer vaccine and the second cancer vaccine differ from each other with respect to at least tumor antigen, formulation, carrier molecule/vehicle, or adjuvant.

### Embodiment 5

The method of embodiment 3, wherein the first cancer vaccine and the second cancer vaccine differ from each other in dose and/or frequency.

### Embodiment 6

The method of any one of embodiments 3-5, wherein the level of tumor-specific GM-CSF response is obtained from a sample obtained from the first subject and the level of tumor-specific GM-CSF response is obtained from a sample obtained from the second subject.

### Embodiment 7

The method of embodiment 2, further comprising administering at least one booster dose of cancer vaccine to the subject if the level of tumor-specific GM-CSF is determined to have diminished.

### Embodiment 8

The method of embodiment 7, wherein said assessing and determining are carried out multiple times over time, and wherein one or more booster doses of the cancer vaccine are administered to the subject as needed.

### Embodiment 9

The method of any one of embodiments 2, 7, or 8, wherein the sample is obtained from the subject immediately before and/or immediately after the first administration of the cancer vaccine (e.g., within day 0 to day+5), to establish a steady state tumor-specific GM-CSF level, and a further sample is obtained from the subject within 30 days 60 days, 90 days, or 180 days or more after the first administration of the cancer vaccine.

### Embodiment 10

The method of embodiment 2, wherein said determining comprises determining whether the level of tumor-specific GM-CSF has diminished to an extent that is inconsistent with a positive clinical outcome.

### Embodiment 11

The method of embodiment 10, wherein the positive clinical outcome is selected from alleviation of one or more symptoms of the cancer, diminishment of extent of disease, stabilized (i.e., not worsening) state of disease, delay or slowing of disease progression, amelioration or palliation of the disease state, remission (whether partial or total), whether detectable or undetectable, tumor regression, inhibition of tumor growth, inhibition of tumor metastasis, reduction in cancer cell number, inhibition of cancer cell infiltration into peripheral organs, improved time to disease progression (TTP), improved response rate (RR), prolonged overall survival (OS), prolonged time-to-next-treatment (TNTT), or prolonged time from first progression to next treatment, or a combination of two or more of the foregoing.

### Embodiment 12

A kit for the effective treatment of cancer in a subject, comprising: one or more doses of a cancer vaccine; and one or more reagents for the detection of tumor-specific (anti-tumor) granulocyte-macrophage colony-stimulating factor (GM-CSF).

### Embodiment 13

The kit of embodiment 12, wherein the one or more reagents comprise an antibody directed against GM-CSF or an oligonucleotide probe targeting GM-CSF DNA or mRNA.

### Embodiment 14

A method for qualifying subjects for cancer vaccination, the method comprising assessing the level of granulocyte-macrophage colony-stimulating factor (GM-CSF) in a sample of the subject, wherein cancer vaccination is authorized if the level of GM-CSF in the sample is consistent with effective treatment, and wherein cancer vaccination is not authorized if the level of GM-CSF in the sample is not consistent with effective treatment.

### Embodiment 15

The method of embodiment 14, wherein the subject has cancer and the subject is under consideration for administration of a cancer vaccine, wherein administration of the cancer vaccine is authorized if the level of GM-CSF in the sample is consistent with effective treatment for the cancer, and wherein administration of the cancer vaccine is authorized if the level of GM-CSF in the sample is not consistent with effective treatment for the cancer.

### Embodiment 16

A method for treating cancer in a subject by inducing a granulocyte-macrophage colony-stimulating factor (GM-CSF) response against a cancer in the subject, the method comprising administering an effective amount of the cancer vaccine to the subject to induce the GM-CSF response.

### Embodiment 17A

The method of embodiment 16, wherein the cancer vaccine comprises a tumor-specific antigen (TSA) or tumor-associated antigen (TAA), and wherein the method comprises administering an effective amount of the cancer vaccine to the subject to induce a GM-CSF response against the TSA or TAA.

### Embodiment 17B

The method of embodiment 16 or 17B, further comprising assessing the GM-CSF response against the cancer in the subject one or more times before, during, and/or after administering the cancer vaccine to the subject.

### Embodiment 18

The method of any preceding embodiment, wherein the outcome of treatment is overall survival (OS).

### Embodiment 19

The method of any preceding embodiment, wherein the outcome of treatment is time-to-next-treatment (TNTT).

### Embodiment 20

The method of any preceding embodiment, wherein the outcome of treatment is time from first progression to next treatment.

### Embodiment 22

The method or kit of any preceding embodiment, wherein the sample comprises or consists of peripheral blood mononuclear cells (PBMCs) or supernatant from a culture of the PBMCs.

### Embodiment 23

The method or kit of any preceding embodiment, wherein the sample comprises or consists of helper T-cells or supernatant from a culture of the helper T-cells.

### Embodiment 24

The method of any preceding embodiment, wherein the reference level of GM-CSF is a range.

### Embodiment 25

The method of any one of embodiments 1-23, wherein the reference level of GM-CSF is a threshold or cutoff value.

### Embodiment 26

The method of any preceding embodiment, wherein the subject has been previously treated with the cancer vaccine before the sample is obtained from the subject.

### Embodiment 27

The method of any preceding embodiment, wherein the subject has been previously treated with the cancer vaccine, and wherein said method further comprises administering a different treatment for the cancer if the prognosticated outcome of treatment with the cancer vaccine is not desirable.

### Embodiment 28

The method of any preceding embodiment, wherein the sample is obtained from the subject immediately before and/or immediately after the first administration of the cancer vaccine (e.g., within day 0 to day+5), to establish a steady state tumor-specific GM-CSF level, and a further sample is obtained from the subject within 30 days 60 days, 90 days, or 180 days or more after the first administration of the cancer vaccine.

### Embodiment 29

The method of any one of embodiments 1-19, wherein the subject has not been treated with the cancer vaccine before the sample is obtained from the subject.

### Embodiment 30

The method of embodiment 1, further comprising determining the level of tumor-specific GM-CSF in the sample prior to said comparing.

### Embodiment 31

The method or kit of any preceding embodiment, wherein the level of tumor-specific GM-CSF is determined using an immunoassay.

### Embodiment 32

The method or kit of any preceding embodiment, wherein the level of tumor-specific GM-CSF is determined using a competitive or immunometric assay.

### Embodiment 33

The method or kit of embodiment 32, wherein the competitive or immunometric assay is radioimmunoassay (RIA), immunoradiometric assay (IRMA), enzyme-linked immunosorbent assay (ELISA), or enzyme-linked immunosorbent spot (ELISPOT) assay.

### Embodiment 34

The method or kit of any preceding embodiment, wherein the level of tumor-specific GM-CSF is determined by determining the level of tumor-specific GM-CSF mRNA.

### Embodiment 35

The method or kit of any preceding embodiment, wherein the level of tumor-specific GM-CSF is determined by determining the level of tumor-specific GM-CSF protein.

### Embodiment 36

The method or kit of any preceding embodiment, wherein the level of tumor-specific GM-CSF is determined using surface plasmon resonance, fluorescence resonance energy transfer, bioluminescence resonance energy transfer, fluorescence quenching flurorescence, fluorescence polarization, mass spectrometry (MS), high-performance liquid chromatography (HPLC), high-performance liquid chromatography/mass spectrometry (HPLC/MS), high-performance liquid chromatography/mass spectrometry/mass spectrometry (HPLC/MS/MS), capillary electrophoresis, rod-gel electrophoresis, or slab-gel electrophoresis.

### Embodiment 37

The method embodiment 1 or 3, further comprising obtaining the sample from the subject prior to the comparing step.

### Embodiment 38

The method or kit of any preceding embodiment, wherein the cancer is a B-cell malignancy.

### Embodiment 39

The method or kit of embodiment 38, wherein the B-cell malignancy is selected from the group consisting of non-Hodgkin's lymphoma, chronic lymphocytic leukemia (CLL), small lymphocytic lymphoma, multiple myeloma, mantle cell lymphoma, B-cell prolymphocytic leukemia, lymphoplasmocytic lymphoma, splenic marginal zone lymphoma, marginal zone lymphoma (extra-nodal and nodal), follicular lymphoma (grades I, II, III, or IV), diffuse large B-cell lymphoma, mediastinal (thymic) large B-cell lymphoma, intravascular large B-cell lymphoma, primary effusion lymphoma, Burkitt lymphoma/leukemia.

### Embodiment 40

The method or kit of embodiment 38, wherein the B-cell malignancy is a mature B-cell lymphoma.

### Embodiment 41

The method or kit of embodiment 38, wherein the B-cell malignancy is a mature B-cell lymphoma selected from the group consisting of B-cell chronic lymphocytic leukemia/small lymphocytic lymphoma, B-cell prolymphocytic leukemia, lymphoplasmacytic lymphoma, splenic marginal zone B-cell lymphoma (½ villous lymphocytes), hairy cell leukemia, plasma cell myeloma/plasmacytoma, extranodal marginal zone B-cell lymphoma of MALT type, nodal marginal zone B-cell lymphoma (½ monocytoid B cells), follicular lymphoma, mantle-cell lymphoma, diffuse large B-cell lymphoma, mediastinal large B-cell lymphoma, primary effusion lymphoma, Burkitt lymphoma/Burkitt cell leukemia.

### Embodiment 42

The method or kit of embodiment 38, wherein the B-cell malignancy is mantle cell lymphoma or follicular lymphoma.

### Embodiment 43

The method or kit of any preceding embodiment, wherein the cancer vaccine is an autologous idiotype vaccine.

### Embodiment 44

The method or kit of embodiment 43, wherein the autologous idiotype vaccine is hybridoma-derived (such as that described in Example 1).

### Embodiment 45

The method or kit of any preceding embodiment, wherein the cancer vaccine is selected from among a peptide vaccine (such as a tumor-associated or tumor-specific antigen), gene expression vaccine (e.g., plasmid DNA vaccine), recombinant viral vector, recombinant bacteria, dendritic cell vaccine, tumor cell vaccine, heat-shock protein, or exosome-based vaccine.

### Embodiment 46

The method of any preceding embodiment, wherein the cancer treatment further comprises an additional cancer treatment.

### Embodiment 47

The method of embodiment 46, wherein the additional cancer treatment comprises chemotherapy, radiation, immunotherapy, or a combination of two or more of the foregoing.

### Embodiment 48

The method or kit of any preceding embodiment, wherein the subject has previously undergone a different therapy for treatment of the cancer.

### Embodiment 49

The method or kit of embodiment 48, wherein the different therapy comprises chemotherapy and/or immunotherapy.

### Embodiment 50

The method or kit of embodiment 49, wherein the different therapy comprises administration of a monoclonal antibody.

### Embodiment 51

The method or kit of embodiment 50, wherein the different therapy comprises a radioimmunotherapy.

### Embodiment 52

The method or kit of embodiment 48, wherein the different therapy comprises a regimen of PACE (prednisone, doxorubicin, cyclophosphamide, and etoposide), CHOP (cyclophosphamide, doxorubicin, vincristine, and prednisone), CHOP-R (cyclophosphamide, doxorubicin, vincristine, prednisone, rituximab), B-R (bendamustine and rituximab), CVP (cyclophosphamide, vincristine, and prednisone), CVP-R (cyclophosphamide, vincristine, prednisone, and rituximab), F-R (fluradarabine and rituximab), FND-R (fludarabine, mitoxantrone, dexamethasone, and rituximab), FCM (fludarabine, cyclophosphamide, and mitoxantrone), FCM-R (fludarabine, cyclophosphamide, mitoxantrone, and rituximab), radioimmunotherapy, single agent rituximab, single agent alkylator, lenalidomide, involved field radiation therapy, or stem cell transplant.

### Embodiment 53

The method or kit of any preceding embodiment, wherein the subject has undergone an immunochemotherapy induction regimen for treatment of the cancer.

### Embodiment 54

The method or kit of embodiment 53, wherein the immunochemotherapy induction regimen comprises administration of a monoclonal antibody directed against CD20 antigen, and chemotherapy.

### Embodiment 55

The method or kit of embodiment 50 or 54, wherein the monoclonal antibody is rituximab.

### Embodiment 56

The method of embodiment 53, wherein the immunochemotherapy induction regimen is a rituximab-containing combination chemotherapy comprises or consists of etoposide, vincristine, doxorubicin, cyclophosphamide, prednisone, and ritruximab (EPOCH-R).

### Embodiment 57

The method or kit of any preceding embodiment, wherein the subject is in complete remission at the time the cancer vaccine is administered.

### Embodiment 58

The method or kit of any preceding embodiment, wherein the subject is human.

### Embodiment 59

The method or kit of any one of embodiments 1-57, wherein the subject is an animal model.

All patents, patent applications, provisional applications, and publications referred to or cited herein, supra or infra, are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1

### ID-Vaccine Induced GM-CSF Cytokine Improves Overall Survival and Time-to-Next Treatment in Mantle Cell Lymphoma

### BACKGROUND

Some murine lymphoma models show Id-vaccine can induce an antitumor humoral response but others indicate that tumor eradication requires a CD4+ and/or a CD8+ T-cell response. Antitumor T-cells may produce one or many cytokines. Thl/Tcl cytokines (IFNγ, IL-2, TNFα, GM-CSF) are commonly believed to mediate antitumor effects. However, a recent paper (Codarri, L. et al., Nat Immunol., 2011 June; 12(6):560-7. Epub 2011 Apr. 24) proposes that production of GM-CSF by helper T-cells relies on activation of RORγt and its secretion is required for induction of autoimmune inflammation irrespective of helper T-cell polarization. We reported the results of Id-vaccine following DA-EPOCH-R in 26 untreated MCL patients and found no association between PFS (19%) or OS (89%) and immune response at 46-month median potential follow-up. After an 11-year follow-up, we found a highly statistically-significant association between OS and Id-vaccine induced GM-CSF immune responses as well as between Id-vaccine induced GM-CSF immune responses and time-to-next treatment (TTNT).

Pre- and post-vaccine samples were tested in parallel to assess humoral and cellular immune responses. To assess antitumor responses by cytokine induction assay, PBMCs cultured with and without autologous tumor cells. After 6 days, INFα, IFNγ, and GM-CSF were assessed in culture supernatants by ELISA. Normalized post-vaccine responses were calculated for each patient. Characteristics of all 26 patients: median age 57 (range: 22-73), PS 1 (0-2), male sex 73%, blastoid 15%, and MCL international prognostic index (MIPI) (low-65%; intermediary-16%; high-19%). Responses to DA-EPOCHR: CR-92%, PR-8%. Immune analyses were performed in 24 patients; vaccine was not produced in 1 patient and 1 patient progressed before immune analyses.

The associations between OS and MIPI scores and normalized immune responses (KLH and anti-Id antibody responses), frequency of KLH-specific CD4+ T cell responses in PBMC (intracellular IL-2 and TNFα), antitumor cytokine responses, and IFNγ ELISPOT were determined.

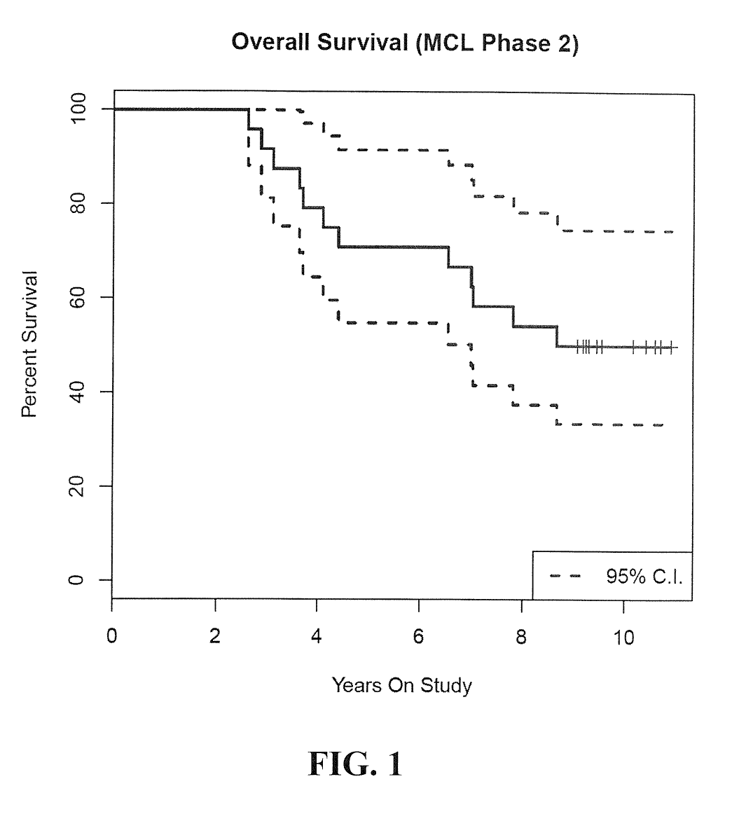

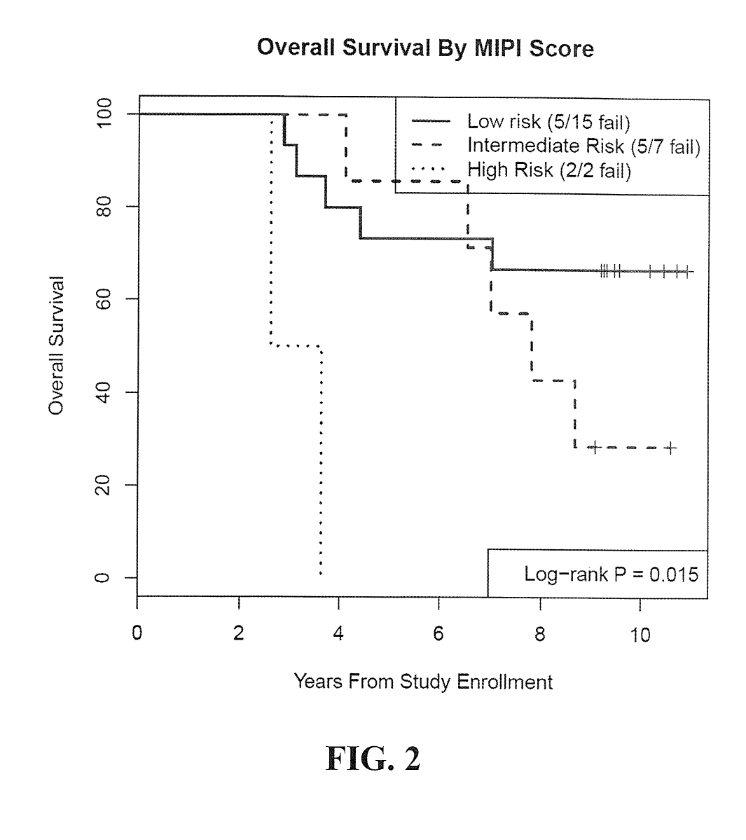

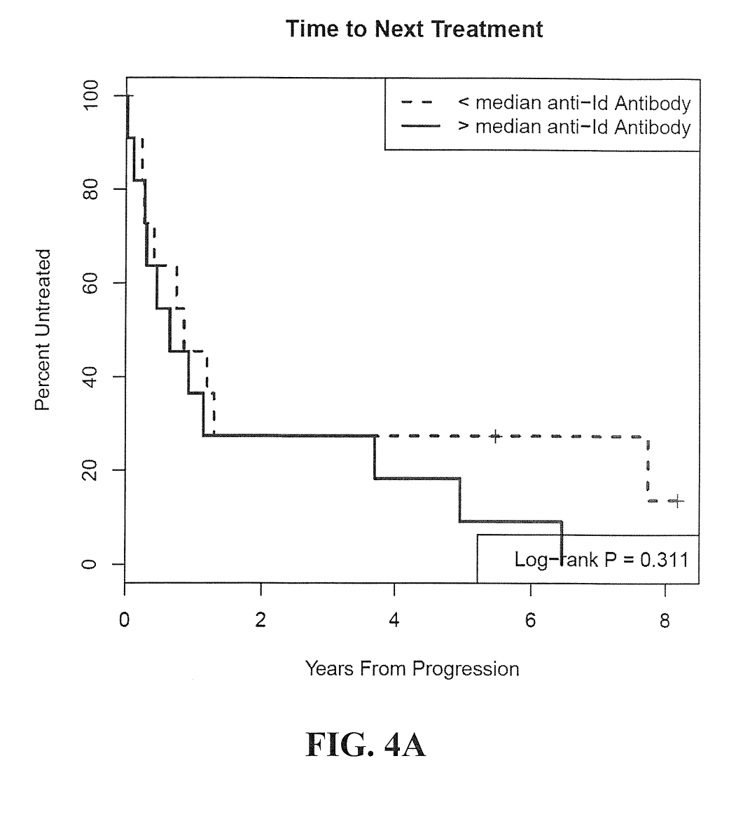

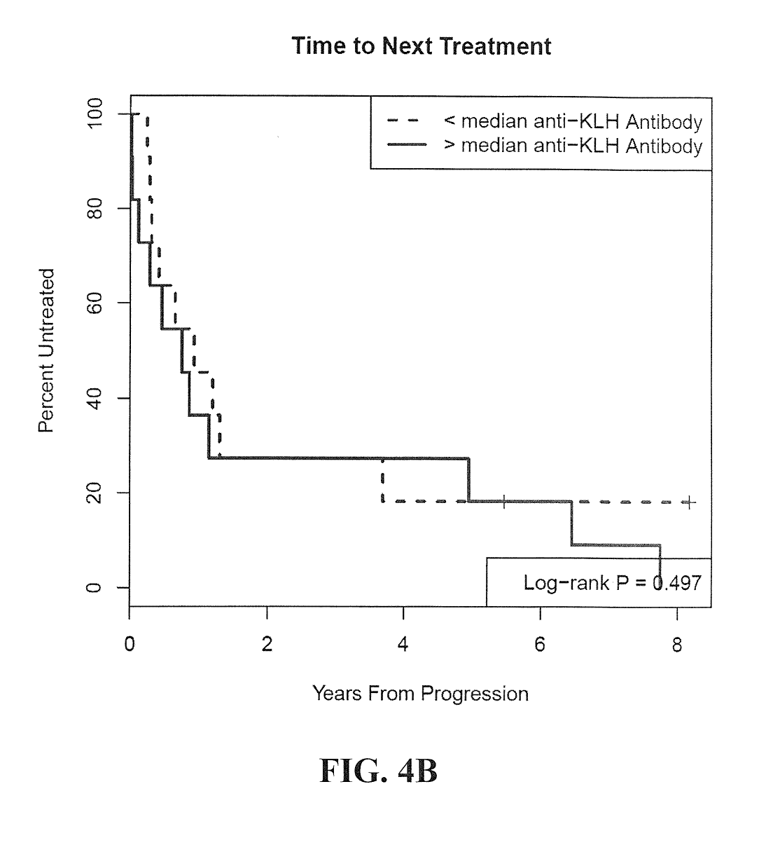

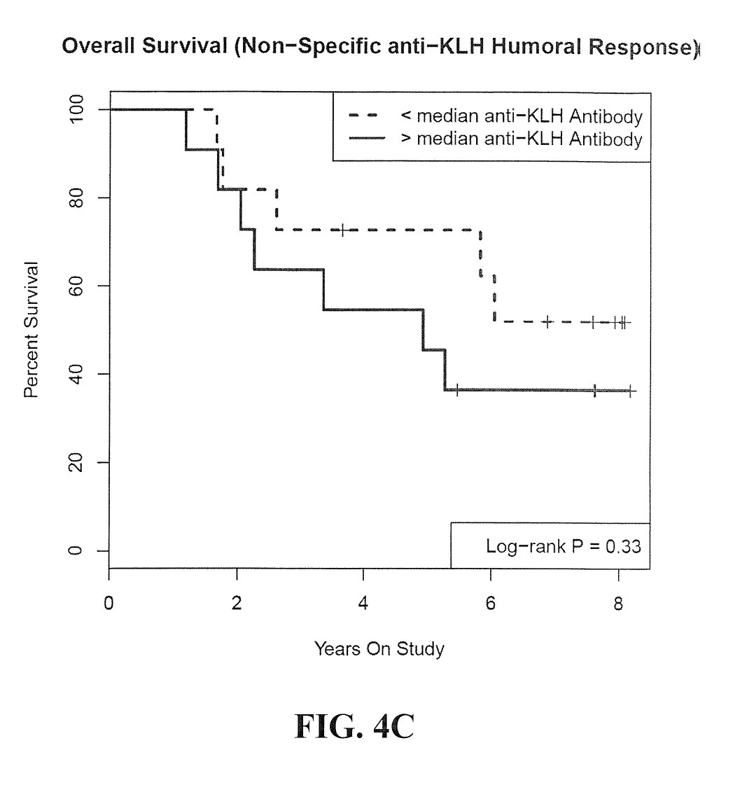

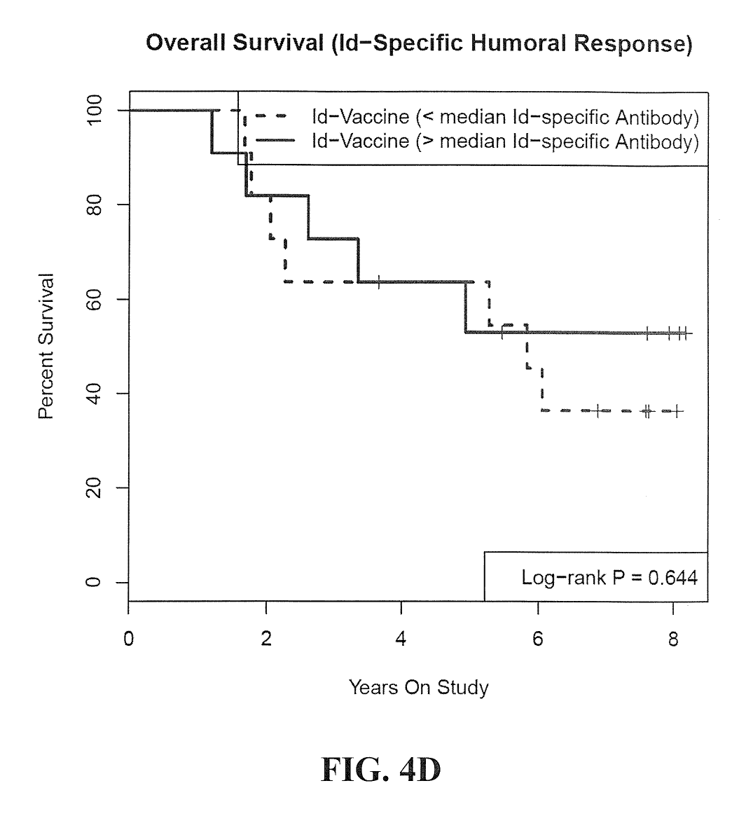

With 122 months median potential follow-up (range: 111-132), the median PFS is 24 months and OS is 104 months (FIG. 1). MIPI was associated with OS (FIG. 2; P=0.01); median OS: low (not reached), intermediary (84 months) and high (44 months). There was no association between OS and KLH humoral response or KLH-specific CD4+ T cells (FIG. 4).

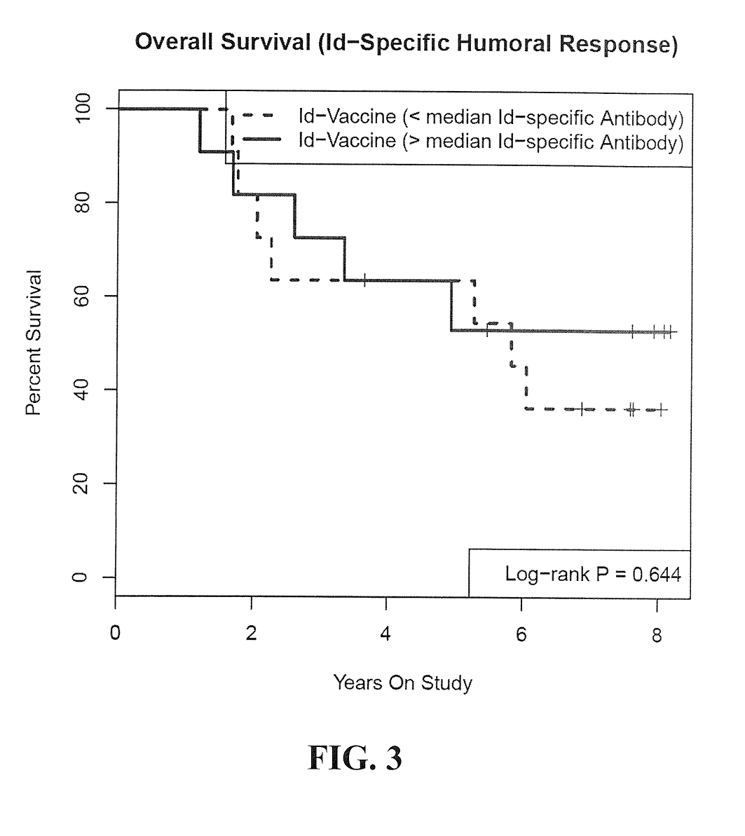

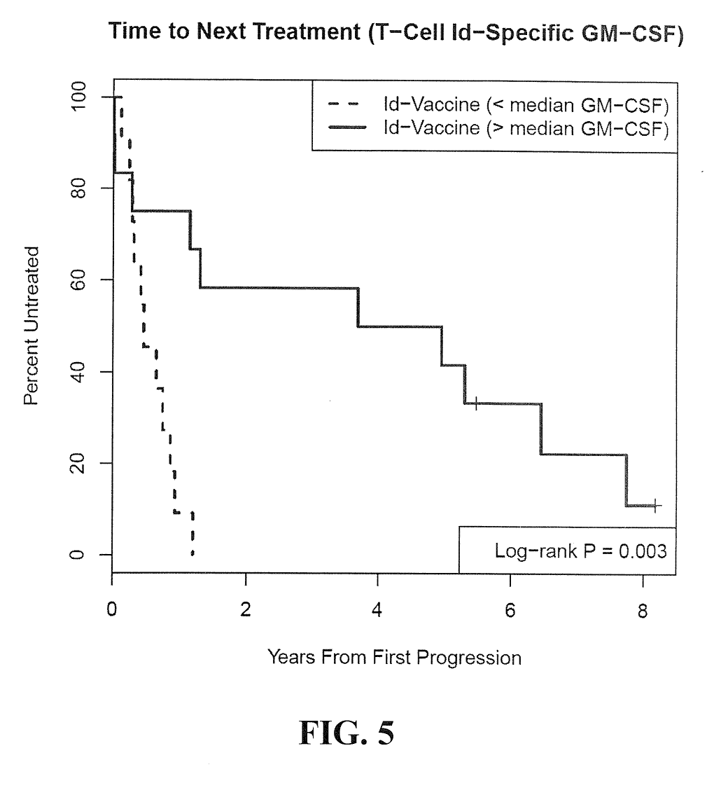

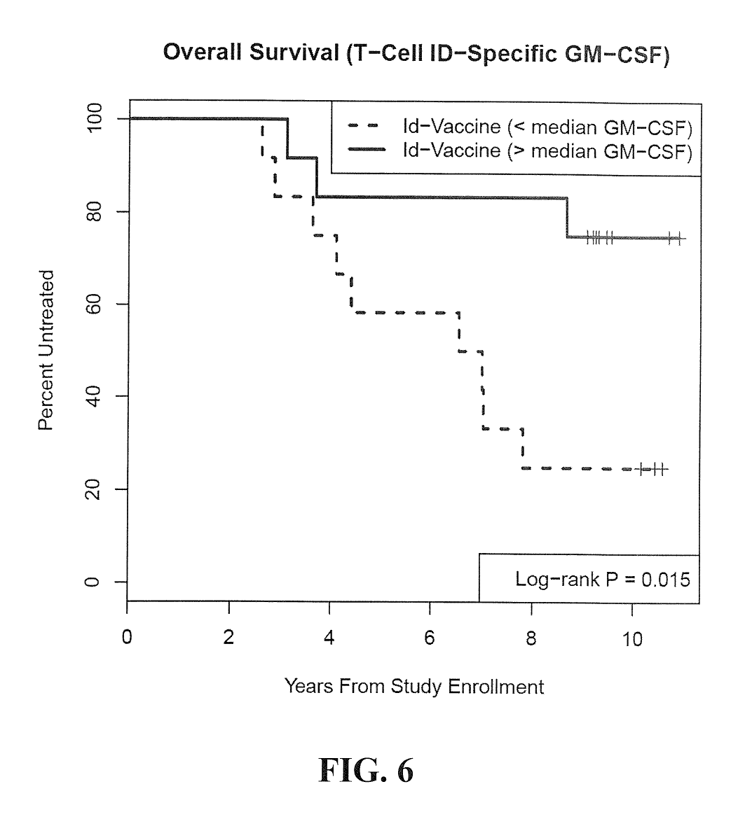

There was also no association between OS and Id-specific humoral response (FIG. 3), IFNγ, ELISPOT, antitumor TNFα, or IFNγ cytokine responses (data not shown). There was a significant association between antitumor GM-CSF production and OS (FIG. 6) as well as a highly significant association between antitumor GM-CSF production and time-to-next-treatment (FIG. 5). There was a significant association between specific antitumor GM-CSF production and OS (FIG. 6); median OS was 79 months versus not reached for patients with values < and > the median GM-CSF value (4.3; p=0.015; p=0.045 adjusted for multiple comparison). GM-CSF immune response was in essence independent of MIPI in a Cox model (p=0.056).

Interestingly, pre-treatment tumor specific-T-cell GM-CSF production correlated with post-vaccine production, suggesting pre-treatment tumor specific T-cell immunity may function in a priming capacity. Importantly, pre-treatment tumor specific immunity did not correlate with OS. We believe these results are the first to suggest that Id-vaccines may improve the survival of MCL, and that survival and not PFS may be the biologically relevant endpoint. We believe this is also the first prospective study to show a significant relationship between a tumor specific immune response and survival following idiotype vaccine in any lymphoma.

With 11 year follow-up, GM-CSF cytokine response mediated by antitumor Tcells significantly correlated with OS. Recent studies support the hypothesis that antitumor T-cells that produce significant amounts of GM-CSF are uniquely polarized and that non-GMCSF producing T-cells do not induce antitumor effects even if they produce TNFα or IFNγ. This may explain why we did not observe an association between OS and TNFα or IFNγ cytokine response or an anti-Id antibody response. These results provide the first evidence that Id-vaccines may improve the survival of MCL following induction with immuno-chemotherapy.

### GM-CSF Cytokine: A Potent T-Cell Activating Cytokine and Potential Mechanism of Action

GM-CSF was first identified as a hematopoietic growth factor to promote the formation of granulocytes and macrophages. Since its discovery, however, its role as a growth factor has been largely recognized as redundant, and instead has been eclipsed by its primary proinflammatory activity (Stanley, E. et al., Proc Natl Acad Sci USA, June 1994, 91(12):5592-5596). Indeed, GM-CSF-deficient mice show evidently normal hematopoiesis but instead do develop pulmonary pathologies (Stanley et al., 1994).

In experimental mouse models, this inflammatory role for this cytokine can perhaps best be understood in models of autoimmune diseases like EAE, myocarditis, and collagen-induced arthritis (McQualter, J. L. et al., J Exp Med, October 2001, 194(7): 873-882; Campbell, I. K. et al., J Immunol, October 1998, 161(7):3639-3644; Sonderegger, Ivo et al., J Exp Med, September, 2008, 205(10): 2281-2294) in these conditions, mice deficient in GM-CSF show marked resistance to the development of each condition. In EAE, Codarri, et al. demonstrated that T cell production of GM-CSF critically impacts the effector phase of EAE (Codarri et al., 2011).

In their study, Codarri, et al. noted a number of key properties of GM-CSF (Codarri et al., 2011):
signaling through the IL-23 receptor complex elicited the secretion of GM-CSF by encephalitogenic helper T cells in vivo and in vitro. [IL-23 functions upstream of GM-CSF]
exposure to IL-23 rendered helper T cells lacking either IFN-γ or IL-17A or both fully able to cause CNS inflammation. [IFN-γ and IL-17A were not necessary for disease induction]
GM-CSF-deficient T cells were completely unable to induce EAE, and even higher ex-pression of IL-17A or IFN-γ failed to compensate for the loss of GM-CSF.
GM-CSF-deficient T cells invaded CNS, but did not accumulate and lead to autoimmunity.

Moreover, IL-23 and GM-CSF appear to be subject to a cross-regulation mechanism, where GM-CSF secreted by TH17 cells stimulates by the production of IL-23 by APCs, which in turn enhances the TH17 differentiation and amplifies the inflammatory response.

These reported findings suggest an important role of GM-CSF in the initiation and propagation of the cell-mediated immune response, and provide a potential underlying explanation of the association between GM-CSF cytokine induction and survival in the MCL study.

### Idiotype Vaccine Efficacy and Benefit

The results of these clinical studies indicate that the vaccine composed of autologous lymphomaderived ID conjugated to keyhole limpet hemocyanin (KLH) has biologic efficacy, clinical efficacy, and clinical benefit. The studies establish that Id-vaccination elicits specific immune responses, including tumor-specific cellular, cytokine and anti-idiotypic antibody responses (Barrios, Y. et al., Haematologica, 2002, 87(4):400-407; Hsu, F J et al., Blood, May 1997, 89(9):3129-3135; Inoges, S. et al., J Natl Cancer Inst, 2006, 98(18):1292-301; Kwak, L W et al., The New England Journal of Medicine, October 1992, 327(17): 1209-1215; Weng, W. K. et al., J Clin Oncol, December, 2004, 22(23):4717-24; Bendandi, M. et al., Nat Med, October 1999, 5(10): 1171-7). This effect persists even when vaccination follows severe B-cell immunosuppression due to rituximab (Neelapu, S. et al., Clin Lymphoma, 2005, 6(1): 61-4). Moreover, direct evidence of the Id vaccine's ability to induce tumor cell clearance was established by the clearance of tumor cells from the peripheral blood (Bendandi et al., 1999; Barrios et al., 2002).

In the Phase 2 studies, Id vaccination demonstrated early signals that treatment could extend key clinical endpoints, such as DFS or OS, was demonstrated by the association between induction of immune responses and improved DFS (F. Hsu et al., 1997; Inoges et al., 2006; Weng et al., 2004; Nelson, E. L. et al., Blood, July 1996, 88(2):580-9).

1. A method of prognosticating an outcome of cancer treatment with a cancer vaccine in a subject, comprising comparing the level of tumor-specific (anti-tumor) granulocyte-macrophage colony-stimulating factor (GM-CSF) in a sample obtained from the subject with a reference level of GM-CSF, wherein the level of tumor-specific GM-CSF in the sample compared to the reference level of GM-CSF is prognostic for an outcome of treatment with the cancer vaccine.

2-7. (canceled)

8. The method of claim 1, wherein the subject has been previously treated with the cancer vaccine before the sample is obtained from the subject.

9. The method of claim 1, wherein the subject has been previously treated with the cancer vaccine, and wherein said method further comprises administering a different treatment for the cancer if the prognosticated outcome of treatment with the cancer vaccine is not desirable.

10. The method of claim 8, wherein the sample is obtained from the subject immediately before and/or immediately after the first administration of the cancer vaccine (e.g., within day 0 to day+5), to establish a steady state tumor-specific GM-CSF level, and a further sample is obtained from the subject within 30 days 60 days, 90 days, or 180 days or more after the first administration of the cancer vaccine.

11-23. (canceled)

24. The method of claim 1, wherein cancer is mantle cell lymphoma or follicular lymphoma.

25. The method of claim 1, wherein the cancer vaccine is an autologous idiotype vaccine.

26. The method of claim 1, wherein the cancer vaccine is selected from among a peptide vaccine, plasmid DNA vaccine, recombinant viral vector, recombinant bacteria, dendritic cell vaccine, tumor cell vaccine, heat-shock protein, or exosome-based vaccine.

27-28. (canceled)

29. The method of claim 1, wherein the subject has previously undergone a different therapy for treatment of the cancer.

30-32. (canceled)

33. The method of claim 29, wherein the different therapy comprises a regimen of PACE (prednisone, doxorubicin, cyclophosphamide, and etoposide), CHOP (cyclophosphamide, doxorubicin, vincristine, and prednisone), CHOP-R (cyclophosphamide, doxorubicin, vincristine, prednisone, rituximab), B-R (bendamustine and rituximab), CVP (cyclophosphamide, vincristine, and prednisone), CVP-R (cyclophosphamide, vincristine, prednisone, and rituximab), F-R (fluradarabine and rituximab), FND-R (fludarabine, mitoxantrone, dexamethasone, and rituximab), FCM (fludarabine, cyclophosphamide, and mitoxantrone), FCM-R (fludarabine, cyclophosphamide, mitoxantrone, and rituximab), radioimmunotherapy, single agent rituximab, single agent alkylator, lenalidomide, involved field radiation therapy, or stem cell transplant.

34-40. (canceled)

41. A method selected from among:
(a) a method for treating cancer in a subject with a cancer vaccine, the method comprising: assessing the level of tumor-specific (anti-tumor) granulocyte-macrophage colony-stimulating factor (GM-CSF) in a sample obtained from a subject that has been administered a cancer vaccine for treatment of a cancer; and determining whether the level of tumor-specific GM-CSF has diminished in the subject; or
(b) a method for qualifying subjects for cancer vaccination, the method comprising assessing the level of granulocyte-macrophage colony-stimulating factor (GM-CSF) in a sample of the subject, Wherein cancer vaccination is authorized if the level of GM-CSF in the sample is consistent with effective treatment, and wherein cancer vaccination is not authorized if the level of GM-CSF in the sample is not consistent with effective treatment; or
(c) a method for treating cancer in a subject by inducing a granulocyte-macrophage colony-stimulating factor (GM-CSF) response against a cancer in the subject, the method comprising administering an effective amount of a cancer vaccine to the subject to induce the GM-CSF response; or
(d) a method for comparing the efficacy of two or more cancer vaccine treatments, comprising comparing the level of tumor-specific (anti-tumor) granulocyte macrophage colony-stimulating factor (GM-CSF) response from a subject that has received a first cancer vaccine treatment to the level of GM-CSF response from a subject that has received a second cancer vaccine treatment, wherein the first vaccine cancer treatment and the second cancer vaccine treatment differ in cancer vaccine composition and/or in cancer vaccine administration.

42. The method of claim 41, wherein the method is (a), further comprising administering at least one booster dose of cancer vaccine to the subject if the level of tumor-specific GM-CSF is determined to have diminished.

43. The method of claim 42, wherein said assessing and determining are carried out multiple times over time, and wherein one or more booster doses of the cancer vaccine are administered to the subject as needed.

44. The method of claim 41, wherein the method is (a), and wherein the sample is obtained from the subject immediately before and/or immediately after the first administration of the cancer vaccine (e.g., within day 0 to day+5), to establish a steady state tumor-specific GM-CSF level, and a further sample is obtained from the subject within 30 days 60 days, 90 days, or 180 days or more after the first administration of the cancer vaccine.

45. The method of claim 41, wherein the method is (a), and wherein said determining comprises determining whether the level of tumor-specific GM-CSF has diminished to an extent that is inconsistent with a positive clinical outcome.

46. (canceled)

47. A kit for the effective treatment of cancer, comprising: one or more doses of a cancer vaccine; and one or more reagents for the detection of tumor-specific (anti-tumor) granulocyte-macrophage colony-stimulating factor (GM-CSF).

48-49. (canceled)

50. The method of claim 41, wherein the method is (b), wherein the subject has cancer and the subject is under consideration for administration of a cancer vaccine, wherein administration of the cancer vaccine is authorized if the level of GM-CSF in the sample is consistent with effective treatment for the cancer, and wherein administration of the cancer vaccine is authorized if the level of GM-CSF in the sample is not consistent with effective treatment for the cancer.

51. (canceled)

52. The method of claim 41, wherein the method is (c), wherein the cancer vaccine comprises a tumor-specific antigen (ISA) or tumor-associated antigen (TAA), and wherein the method comprising administering an effective amount of the cancer vaccine to the subject to induce a GM-CSF response against the TSA or TAA.

53. The method of claim 41, wherein the method is (c), further comprising assessing the GM-CSF response against the cancer in the subject one or more times before, during, and/or after administering the cancer vaccine to the subject.

54. (canceled)

55. The method of claim 51, wherein the method is (d), and wherein the first cancer vaccine and the second cancer vaccine differ from each other with respect to at least tumor antigen, formulation, carrier molecule/vehicle, or adjuvant.

56. The method of claim 51, wherein the method is (d), and wherein the first cancer vaccine and the second cancer vaccine differ from each other in dose and/or frequency.
